(19) United States
(12) Patent Application Publication
(10) Pub. No.: US 2021/0104331 A1
(43) Pub. Date: Apr. 8, 2021

(54) SYSTEMS AND METHODS FOR SCREENING COMPOUNDS IN SILICO

(71) Applicant: Atomwise Inc., San Francisco, CA (US)

(72) Inventors: Venkatesh Mysore, Castro Valley, CA (US); Jon Sorenson, Alameda, CA (US); Greg Friedland, San Francisco, CA (US); Tushita Gupta, Dublin, CA (US); Izhar Wallach, Albany, CA (US)

(21) Appl. No.: 17/038,473

(22) Filed: Sep. 30, 2020

Related U.S. Application Data
(60) Provisional application No. 62/910,068, filed on Oct. 3, 2019.

(51) Int. Cl. G16H 50/70; G16H 70/40; G06N 20/00; G06T 15/10; G16B 5/20; G16B 40/00

(52) U.S. Cl. CPC G16H 50/70; G16H 70/40; G16B 40/00; G06T 15/10; G16B 5/20; G06N 20/00

(57) ABSTRACT

Systems and methods for reducing a number of test objects in a test object dataset are provided. A target model with a first computational complexity is applied to a subset of test objects from the test object dataset and a target object, thereby obtaining a subset of target results. A predictive model with a second computational complexity is trained using the subset of test objects and the subset of target results. The predictive model is applied to the plurality of test objects, thereby obtaining a plurality of predictive results. A portion of the test objects are eliminated from the plurality of test objects based at least in part on the plurality of predictive results. The method determines whether one or more predefined reduction criteria are satisfied. When the predefined reduction criteria are not satisfied, an additional subset of test objects and target results are obtained, and the method is repeated.

### CROSS REFERENCE TO RELATED APPLICATION

This application claims priority to U.S. Provisional Patent Application No. 62/910,068 entitled “Systems and Methods for Screening Compounds In Silico,” filed Oct. 3, 2019, which is hereby incorporated by reference.

### TECHNICAL FIELD

This specification relates generally to techniques for dataset reduction by using multiple computational models with different computational complexities.

### BACKGROUND

The need to diversify molecular scaffolds to improve the chances of success in drug discovery has been referred to as escaping from ‘flatland’—the reliance on synthetic methods that build flat molecules. Another way to investigate the unexplored potential in the molecular universe is to find a way to reveal what is hidden in the shadows. Some estimates say that there are at least 10⁶⁰ different drug-like molecules: a novemdecillion of possibilities. One approach to opening up this dark chemical space is to study ultra-large virtual libraries, that is libraries of compounds that have not necessary been synthesized, but whose molecular properties can be deduced from their calculated molecular structure.

The application of classifiers, such as deep learning neural networks, can be used to generate novel insights from large volumes of data, such as these virtual libraries. Indeed, lead identification and optimization in drug discovery, support in patient recruitment for clinical trials, medical image analysis, biomarker identification, drug efficacy analysis, drug adherence evaluation, sequencing data analysis, virtual screening, molecule profiling, metabolomic data analysis, electronic medical record analysis and medical device data evaluation, off-target side-effect prediction, toxicity prediction, potency optimization, drug repurposing, drug resistance prediction, personalized medicine, drug trial design, agrochemical design, material science and simulations are all examples of applications where the use of classifiers, such as deep learning based solutions, are being explored. Specifically, in health care, the American Recovery and Reinvestment Act of 2009 and the Precision Medicine Initiative of 2015 have widely endorsed the value of medical data in healthcare. Owing to several such initiatives, the amount of medical big data is expected to grow approximately 50-fold to reach 25,000 petabytes by 2020. See e.g., Roots Analysis, Feb. 22, 2017, “Deep Learning in Drug Discovery and Diagnostics, 2017-2035,” available on the Internet at rootsanalysis.com.

With advances in drug repurposing and preclinical research, the application of classifiers to drug discovery has the opportunity to greatly improve drug discovery processes and thus improve patient outcomes throughout the healthcare system. See e.g., Rifaioglu et al., 2018, “Recent applications of deep learning and machine intelligence on in silico drug discovery: methods, tools and databases,” Briefings in Bioinform 1-35; and Lavecchia, 2015, “Machine-learning approaches in drug discovery: methods and applications,” Drug Discovery Today 20(3), 318-331. Methods of in silico drug discovery are particularly valuable applications of classifiers as these have the potential to reduce the time and expense of drug development. Currently, the average cost of developing a new drug for use in humans is estimated to be well over $2 billion. See e.g., DiMasi et al., 2016, J Health Econ 47, 20-33. In addition, the United States federal government, largely through NIH funding, spent more than $100 billion on primarily basic research that contributed to all of the 210 new drugs approved by the FDA from 2010-2016. See Cleary et al., 2018, “Contributions of NIH funding to new drug approvals 2010-2016,” PNAS 115(10), 2329-2334. Thus, computational methods to discover or at least screen for (e.g., in databases of known and/or FDA approved chemicals) lead compounds have the potential to revolutionize drug discovery and development.

There are many examples of computational methods aiding drug discovery. The discovery of polypharmacology (e.g., the understanding that many drugs can and do bind to more than one molecular target) opened the field of repurposing already approved drugs for diseases that lacked treatments. See e.g., Hopkins, 2009, “Predicting promiscuity,” Nature 462, 167-168 and Keiser et al., 2007, “Relating protein pharmacology by ligand chemistry,” Nat Biotechnol 25(2), 197-206. In silico drug discovery has already produced potential treatments for diseases ranging from Zika to Chagas disease. See e.g., Ramarack et al., 2017, “Zika virus NS5 protein potential inhibitors: an enhanced in silico approach in drug discovery,” J Biomol Structure and Dynamics 36(5), 1118-1133; Castillo-Garit et al., 2012, “Identification in silico and in vitro of Novel Trypanosomicidal Drug-Like Compounds,” Chem Biol and Drug Des 80, 38-45; and Raj et al. 2015 “Flavonoids as Multi-target Inhibitors for Proteins associated with Ebola Virus,” Interdisip Sci Comput Life Sci 7, 1-10. However, one drawback with many of the methods used currently for drug discovery, including the evaluation of virtual libraries, is their computational complexity.

In particular, many in silico drug discovery methods are applicable primarily to pre-filtered and size-restricted molecular databases. See e.g., Macalino et al., 2018, “Evolution of in Silico Strategies for Protein-Protein Interaction Drug Discovery,” Molecules 23, 1963 and Lionata et al., 2014, “Structure-Based Virtual Screening for Drug Discovery: Principles, Applications and Recent Advances,” Curr Top Med Chem 14(16): 1923-1938. In particular, datasets are typically restricted to at least the low millions of compounds. See Ramsundar et al., 2015, “Massively Multitask Networks for Drug Discovery,” arXiv:1502.02072. The limitations on database size impose corresponding limitations on the ability to discover or screen for drugs with the potential to treat new diseases.

Given the importance of identifying promising lead compounds, improved computational methods of drug discovery that permit evaluation of large libraries of compounds are needed in the art.

### SUMMARY

The present disclosure addresses the shortcomings identified in the background by providing methods for the evaluation of large chemical compound databases.

In one aspect of the present disclosure, a method for reducing a number of test objects in a plurality of test objects in a test object dataset is provided. The method comprises obtaining, in electronic format, the test object dataset.

The method further comprises applying a target model, for each respective test object in a subset of test objects from the plurality of test objects, to the respective test object and at least one target object to obtain a corresponding target result, thereby obtaining a corresponding subset of target results.

The method further trains a predictive model in an initial trained state using at least i) the subset of test objects as independent variables of the predictive model and ii) the corresponding subset of target results as dependent variables of the predictive model, thereby updating the predictive model to an updated trained state.

The method further applies the predictive model in an updated trained state to the plurality of test objects thereby obtaining an instance of a plurality of predictive results.

The method further eliminates a portion of the test objects from the plurality of test objects based at least in part on the instance of the plurality of predictive results.

The method further comprises determining whether one or more predefined reduction criteria are satisfied. When the one or more predefined reduction criteria are not satisfied, the method further comprises (i) applying, for each respective test object in an additional subset of test objects from the plurality of test objects, the target model to the respective test object and at least one target object to obtain a corresponding target result, thereby obtaining an additional subset of target results. The additional subset of test objects is selected at least in part on the instance of the plurality of predictive results. The method further comprises (ii) updating the subset of test objects by incorporating the additional subset of test objects into the subset of test objects, (iii) updating the subset of target results by incorporating the additional subset of target results into the subset of target results, and (iv) modifying, after the updating (ii) and (iii), the predictive model by applying the predictive model to at least 1) the subset of test objects as independent variables and 2) the corresponding subset of target results as corresponding dependent variables, thereby providing the predictive model in an updated trained state. The method then repeats the application of the predictive model in an updated trained state to the plurality of test objects thereby obtaining an instance of a plurality of predictive results. The method further eliminates a portion of the test objects from the plurality of test objects based at least in part on the instance of the plurality of predictive results until the one or more predefined reduction criteria are satisfied.

In some embodiments, the target model exhibits a first computational complexity in evaluating test objects, the predictive model exhibits a second computational complexity in evaluating test object, and the second computational complexity is less than the first computational complexity. In some embodiments, the target model is at least three-fold, at least five-fold or at least 100-fold more computationally complex than the predictive model.

In some embodiments, the test object dataset includes a plurality of feature vectors (e.g., protein fingerprints, computational properties, and/or graph descriptors). In some embodiments, each feature vector is for a respective test object in the plurality of test objects, and a size of each feature vector in the plurality of feature vectors is the same. In some embodiments, each feature vector in the plurality of feature vectors is a one-dimensional vector.

In some embodiments, the applying a target model, for each respective test object in a subset of test objects from the plurality of test objects, to the respective test object and at least one target object to obtain a corresponding target result, thereby obtaining a corresponding subset of target results further comprises randomly selecting one or more test objects from the plurality of test objects to form the subset of test objects.

In some embodiments, applying a target model, for each respective test object in a subset of test objects from the plurality of test objects, to the respective test object and at least one target object to obtain a corresponding target result, thereby obtaining a corresponding subset of target results further comprises selecting one or more test objects from the plurality of test objects for the subset of test objects based on evaluation of one or more features selected from the plurality of feature vectors. In some embodiments, the selection is based on clustering (e.g., of the plurality of test objects).

In some embodiments, satisfaction of the one or more predefined reduction criteria comprises comparing each predictive result in the plurality of predictive results to a corresponding target result from the subset of target results. In some embodiments, the one or more predefined reduction criteria are satisfied when the difference between training and target results falls below a predetermined threshold.

In some embodiments, satisfaction of the one or more predefined reduction criteria comprises determining that the number of test objects in the plurality of test objects has dropped below a threshold number of objects.

In some embodiments, the target model is a convolutional neural network.

In some embodiments, the predictive model comprises a random forest tree, a random forest comprising a plurality of multiple additive decision trees, a neural network, a graph neural network, a dense neural network, a principal component analysis, a nearest neighbor analysis, a linear discriminant analysis, a quadratic discriminant analysis, a support vector machine, an evolutionary method, a projection pursuit, a linear regression, a Naïve Bayes algorithm, a multi-category logistic regression algorithm, or ensembles thereof.

In some embodiments, the at least one target object is a single object, and the single object is a polymer. In some embodiments, the polymer comprises an active site. In some embodiments, the polymer is a protein, a polypeptide, a polynucleic acid, a polyribonucleic acid, a polysaccharide, or an assembly of any combination thereof.

In some embodiments, the plurality of test objects, before application of an instance of the eliminating a portion of the test objects from the plurality of test objects, comprises at least 100 million test objects, at least 500 million test objects, at least 1 billion test objects, at least 2 billion test objects, at least 3 billion test objects, at least 4 billion test objects, at least 5 billion test objects, at least 6 billion test objects, at least 7 billion test objects, at least 8 billion test objects, at least 9 billion test objects, at least 10 billion test objects, at least 11 billion test objects, at least 15 billion test objects, at least 20 billion test objects, at least 30 billion test objects, at least 40 billion test objects, at least 50 billion test objects, at least 60 billion test objects, at least 70 billion test objects, at least 80 billion test objects, at least 90 billion test objects, at least 100 billion test objects, or at least 110 billion test objects.

In some embodiments, the one or more predefined reduction criteria require the plurality of test objects (e.g., after one or more instances of the eliminating a portion of the test objects from the plurality of test objects) to have no more than 30 test objects, no more than 40 test objects, no more than 50 test objects, no more than 60 test objects, no more than 70 test objects, no more than 90 test objects, no more than 100 test objects, no more than 200 test objects, no more than 300 test objects, no more than 400 test objects, no more than 500 test objects, no more than 600 test objects, no more than 700 test objects, no more than 800 test objects, no more than 900 test objects, or no more than 1000 test objects.

In some embodiments, each test object in the plurality of test objects is a chemical compound.

In some embodiments, the predictive model in the initial trained state comprises an untrained or partially trained classifier. In some embodiments, the predictive model in the updated trained state comprises an untrained or a partially trained classifier that is distinct from the predictive model in the initial trained state.

In some embodiments, the subset of test objects and/or the additional subset of test objects comprises at least 1,000 test objects, at least 5,000 test objects, at least 10,000 test objects, at least 25,000 test objects, at least 50,000 test objects, at least 75,000 test objects, at least 100,000 test objects, at least 250,000 test objects, at least 500,000 test objects, at least 750,000 test objects, at least 1 million test objects, at least 2 million test objects, at least 3 million test objects, at least 4 million test objects, at least 5 million test objects, at least 6 million test objects, at least 7 million test objects, at least 8 million test objects, at least 9 million test objects, or at least 10 million test objects. In some embodiments, the additional subset of test objects is distinct from the subset of test objects.

In some embodiments, the training a predictive model in an initial trained state using at least i) the subset of test objects as a plurality of independent variables (of the predictive model) and ii) the corresponding subset of target results as a plurality of dependent variables (of the predictive model) further comprises using iii) the at least one target object as an independent variable of the predictive model.

In some embodiments, the at least one target object comprises at least two target objects, at least three target objects, at least four target objects, at least five target objects, or at least six target objects.

In some embodiments, the modifying after the updating (ii) and the updating (iii), the predictive model by applying the predictive model (iv) further comprises using 3) the at least one target object as an independent variable, in addition to using at least 1) the subset of test objects as independent variables and 2) the corresponding subset of target results as corresponding dependent variables.

In some embodiments, when the one or more predefined reduction criteria are satisfied, the method further comprises clustering the plurality of test objects, thereby assigning each test object in the plurality of test objects to a cluster in a plurality of clusters; and eliminating one or more test objects from the plurality of test objects based at least in part on redundancy of test objects in individual clusters in the plurality of clusters.

In some embodiments, the method further comprises selecting the subset of test objects from the plurality of test objects by clustering the plurality of test objects thereby assigning each test object in the plurality of test objects to a respective cluster in a plurality of clusters, and selecting the subset of test objects from the plurality of test objects based at least in part on a redundancy of test objects in individual clusters in the plurality of clusters.

In some embodiments, when the one or more predefined reduction criterion are satisfied, the method further comprises applying the plurality of test objects and the at least one target object to the predictive model thereby causing the predictive model to provide a respective predictive result for each test object in the plurality of test objects. In some embodiments, each respective predictive results corresponds to a prediction of an interaction between a respective test object and the at least one target object (e.g., IC₅₀, EC₅₀, Kd, or KI). In some embodiments, each respective prediction score is used to characterize the at least one target object.

In some embodiments, the eliminating a portion of the test objects from the plurality of test objects based at least in part on the instance of the plurality of predictive results comprises: i) clustering the plurality of test objects, thereby assigning each test object in the plurality of test objects to a respective cluster in a plurality of clusters, and ii) eliminating a subset of test objects from the plurality of test objects based at least in part on a redundancy of test objects in individual clusters in the plurality of clusters.

In some embodiments, the clustering of the plurality of test objects is performed using a density-based spatial clustering algorithm, a divisive clustering algorithm, an agglomerative clustering algorithm, a k-means clustering algorithm, a supervised clustering algorithm, or ensembles thereof.

In some embodiments, the eliminating a portion of the test objects from the plurality of test objects based at least in part on the instance of the plurality of predictive results comprises: i) ranking the plurality of test objects based on the instance of the plurality of predictive results, and ii) removing from the plurality of test objects those test objects in the plurality of test objects that fail to have a corresponding interaction score that satisfies a threshold cutoff.

In some embodiments, the threshold cutoff is a top threshold percentage. In some embodiments, the top threshold percentage is the top 90 percent, the top 80 percent, the top 75 percent, the top 60 percent, or the top 50 percent of the plurality of predictive results.

In some embodiments, each instance of the eliminating a portion of the test objects from the plurality of test objects based at least in part on the instance of the plurality of predictive results eliminates between one tenth and nine tenths of the test objects in the plurality of test objects. In some embodiments, each instance of the eliminating eliminates between one quarter and three quarters of the test objects in the plurality of test objects.

Another aspect of the present disclosure provides a computing system comprising at least one processor and memory storing at least one program to be executed by the at least one processor, the at least one program comprising instructions for reducing a number of test objects in a plurality of test objects in a test object dataset by any of the methods disclosed above.

Still another aspect of the present disclosure provides a non-transitory computer readable storage medium storing at least one program for reducing a number of test objects in a plurality of test objects in a test object dataset. The at least one programs is configured for execution by a computer. The at least one program comprises instructions for performing any of the methods disclosed above.

As disclosed herein, any embodiment disclosed herein when applicable can be applied to any other aspect. Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference. In the event of a conflict between a term herein and a term in an incorporated reference, the term herein controls.

### BRIEF DESCRIPTION OF THE DRAWINGS

The implementations disclosed herein are illustrated by way of example, and not by way of limitation, in the accompanying drawings. The description and drawings are only for the purpose of illustration and as an aid to understanding, and are not intended as a definition of the limits of the systems and methods of the present disclosure. Like reference numerals refer to corresponding parts throughout the drawings.

FIG. 1 is a block diagram illustrating an example of a computing system in accordance with some embodiments of the present disclosure.

FIGS. 2A, 2B, and 2C collectively illustrate examples of flowcharts of methods of reducing a number of test objects in a plurality of test objects in a test object dataset, in accordance with some embodiments of the present disclosure.

FIG. 3 illustrates an example of evaluating a compound library in accordance with some embodiments of the present disclosure.

FIG. 4 is a schematic view of an example test object in two different poses relative to a target object, according to an embodiment of the present disclosure.

FIG. 5 is a schematic view of a geometric representation of input features in the form of a three-dimensional grid of voxels, according to an embodiment of the present disclosure.

FIGS. 6 and 7 are views of two test objects encoded onto a two dimensional grid of voxels, according to an embodiment of the present disclosure.

FIG. 8 is the view of the visualization of FIG. 7, in which the voxels have been numbered, according to an embodiment of the present disclosure.

FIG. 9 is a schematic view of geometric representation of input features in the form of coordinate locations of atom centers, according to an embodiment of the present disclosure.

FIG. 10 is a schematic view of the coordinate locations of FIG. 9 with a range of locations, according to an embodiment of the present disclosure.

### DETAILED DESCRIPTION

The computational effort required for drug discovery has increased in concert with the expansion in size and complexity of drug datasets. In particular, highly accurate models of target molecules has enabled the detection of additional test compounds (e.g., potential lead compounds) that might not have been considered using traditional drug discovery methods. The use of computational compound discovery winnows the exploration space of potential drug databases (e.g., by determining which test compounds are most likely to have the desired effect given a particular target molecule) and further simplifies the downstream process of performing clinical tests to verify good test compounds, which is highly labor- and time-intensive.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for training a reference model to determining a tumor fraction for a subject.

### Definitions

As used herein, the term “clustering” refers to various methods of optimizing the grouping of data points into one or more sets (e.g., clusters), where each data point in a respective set comprises a higher degree of similarity to every other data point in the respective set than to data points not in the respective set. There are a wide variety of clustering algorithms that are suitable for evaluating different types of data. These algorithms include hierarchical models, centroid models, distribution models, density-based models, subspace models, graph-based models, and neural models. These different models each have distinct computational requirements (e.g., complexity) and are suitable for different data types. The application of two separate clustering models to the same dataset frequently results in two different groupings of data. In some embodiments, the repeated application of a clustering model to a dataset results in a different grouping of data each time.

As used herein, the term “feature vector” or “vector” is an enumerated list of elements, such as an array of elements, where each element has an assigned meaning. As such, the term “feature vector” as used in the present disclosure is interchangeable with the term “tensor.” For ease of presentation, in some instances a vector may be described as being one-dimensional. However, the present disclosure is not so limited. A feature vector of any dimension may be used in the present disclosure provided that a description of what each element in the vector represents is defined.

As used herein, the term “polypeptide” means two or more amino acids or residues linked by a peptide bond. The terms “polypeptide” and “protein” are used interchangeably herein and include oligopeptides and peptides. An “amino acid,” “residue” or “peptide” refers to any of the twenty standard structural units of proteins as known in the art, which include imino acids, such as proline and hydroxyproline. The designation of an amino acid isomer may include D, L, R and S. The definition of amino acid includes nonnatural amino acids. Thus, selenocysteine, pyrrolysine, lanthionine, 2-aminoisobutyric acid, gamma-aminobutyric acid, dehydroalanine, ornithine, citrulline, and homocysteine are all considered amino acids. Other variants or analogs of the amino acids are known in the art. Thus, a polypeptide may include synthetic peptidomimetic structures such as peptoids. See Simon et al., 1992, Proceedings of the National Academy of Sciences USA, 89, 9367, which is hereby incorporated by reference herein in its entirety. See also Chin et al., 2003, Science 301, 964; and Chin et al., 2003, Chemistry & Biology 10, 511, each of which is incorporated by reference herein in its entirety.

The terminology used in the present disclosure is for the purpose of describing particular embodiments only and is not intended to be limiting to the invention. As used in the detailed description of the invention and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Exemplary System Embodiments

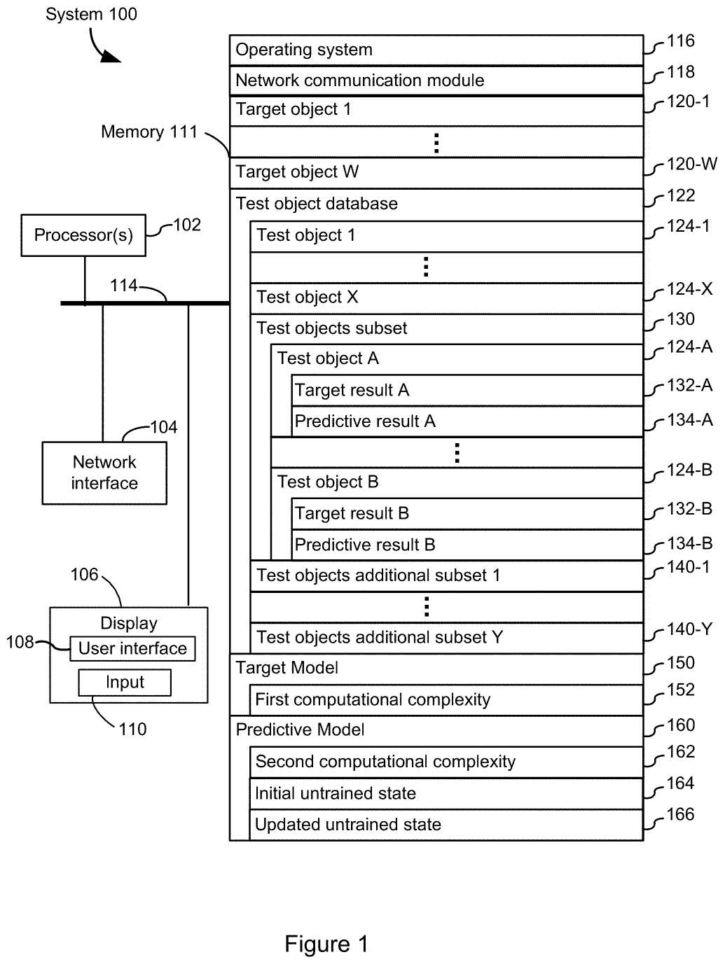

Details of an exemplary system are now described in conjunction with FIG. 1. FIG. 1 is a block diagram illustrating a system 100 in accordance with some implementations. The system 100 in some implementations includes at least one or more processing units CPU(s) 102 (also referred to as processors), one or more network interfaces 104, an optional user interface 108 (e.g., having a display 106, an input device 110, etc.) a memory 111, and one or more communication buses 114 for interconnecting these components. The one or more communication buses 114 optionally include circuitry (sometimes called a chipset) that interconnects and controls communications between system components.

In some embodiments, each processing unit in the one or more processing units 102 is a single-core processor or a multi-core processor. In some embodiments, the one or more processing units 102 is a multi-core processor that enables parallel processing. In some embodiments, the one or more processing units 102 is a plurality of processors (single-core or multi-core) that enable parallel processing. In some embodiments, each of the one or more processing units 102 are configured to execute a sequence of machine-readable instructions, which can be embodied in a program or software. The instructions may be stored in a memory location, such as the memory 111. The instructions can be directed to the one or more processing units 102, which can subsequently program or otherwise configure the one or more processing units 102 to implement methods of the present disclosure. Examples of operations performed by the one or more processing units 102 can include fetch, decode, execute, and writeback. The one or more processing units 102 can be part of a circuit, such as an integrated circuit. One or more other components of the system 100 can be included in the circuit. In some embodiments, the circuit is an application specific integrated circuit (ASIC) or a field-programmable gate array (FPGA) architecture.

In some embodiments, the display 106 is a touch-sensitive display, such as a touch-sensitive surface. In some embodiments, the user interface 106 includes one or more soft keyboard embodiments. In some implementations, the soft keyboard embodiments include standard (QWERTY) and/or non-standard configurations of symbols on the displayed icons. The user interface 106 may be configured to provide a user with graphic showings of, for example, results of reducing a number of test objects in a plurality of test objects in a test object dataset, interaction scores, or predictive results. The user interface may enable user interactions with particular tasks (e.g., reviewing and adjusting predefined reduction criteria).

The memory 111 may be a non-persistent memory, a persistent memory, or any combination thereof. Non-persistent memory typically includes high-speed random access memory, such as DRAM, SRAM, DDR RAM, ROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. Memory 111 optionally includes one or more storage devices remotely located from the CPU(s) 102. Memory 111, and the non-volatile memory device(s) within the memory 111, comprise non-transitory computer readable storage medium. In some embodiments, the memory 111 comprises at least one non-transitory computer readable storage medium, and it stores thereon computer-executable executable instructions which can be in the form of programs, modules, and data structures.

In some embodiments, as shown in FIG. 1, the memory 111 stores the following programs, modules and data structures, or a subset thereof:
instructions, programs, data, or information associated with an operating system 116, (e.g., iOS, ANDROID, DARWIN, RTXC, LINUX, UNIX, OS X, WINDOWS, or an embedded operating system such as VxWorks), which includes various software components and/or drivers for controlling and managing general system tasks (e.g., memory management, storage device control, power management.) and facilitates communication between various hardware and software components;
instructions, programs, data, or information associated with An optional network communication module (or instructions) 118 for connecting the system 100 with other devices and/or to a communication network;
at least one target object 122, where, in some embodiments, the target object comprises a polymer;
a test object database 122 comprising a plurality of test objects 124 (e.g., test objects 124-1, . . . , 124-X), from which a subset 130 of test objects (e.g., test objects 124-A, . . . , 124-B) are selected for analysis by a target model 150, and from which, optionally, one or more additional subsets (e.g., 140-1, . . . , 140-Y) of test objects are selected and subsequently added to subset 130, where each test object 124 in subset 130 has a corresponding target result 132 and a corresponding predictive result 134;
a target model 150 with a first computational complexity 152, where application of the target model to subset 130 of test objects results in a respective target result 132 for each test object 124 in the test object subset 130; and
a predictive model 160 with a second computational complexity 162, where the predicative model, in either an initial 164 or updated 166 untrained state, is applied to test object subset 130 to obtain a respective predictive result 136 for each test object 132 in test object subset 130.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the memory 111 optionally stores a subset of the modules and data structures identified above. Furthermore, in some embodiments, the memory stores additional modules and data structures not described above. In some embodiments, one or more of the above identified elements is stored in a computer system, other than that of system 100, that is addressable by system 100 so that system 100 may retrieve all or a portion of such data when needed.

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items can be separate. Moreover, although FIG. 1 depicts certain data and modules in the memory 111 (which can be non-persistent or persistent memory), it should be appreciated that these data and modules, or portion(s) thereof, may be stored in more than one memory. For example, in some embodiments, at least the first dataset 122, the second dataset 124, the reference module 120, and the reference model 140 are stored in a remote storage device that can be a part of a cloud-based infrastructure. In some embodiments, at least the first dataset 122 and the second dataset 124 are stored on a cloud-based infrastructure. In some embodiments, the reference model 120 and the reference model 140 can also be stored in the remote storage device(s).

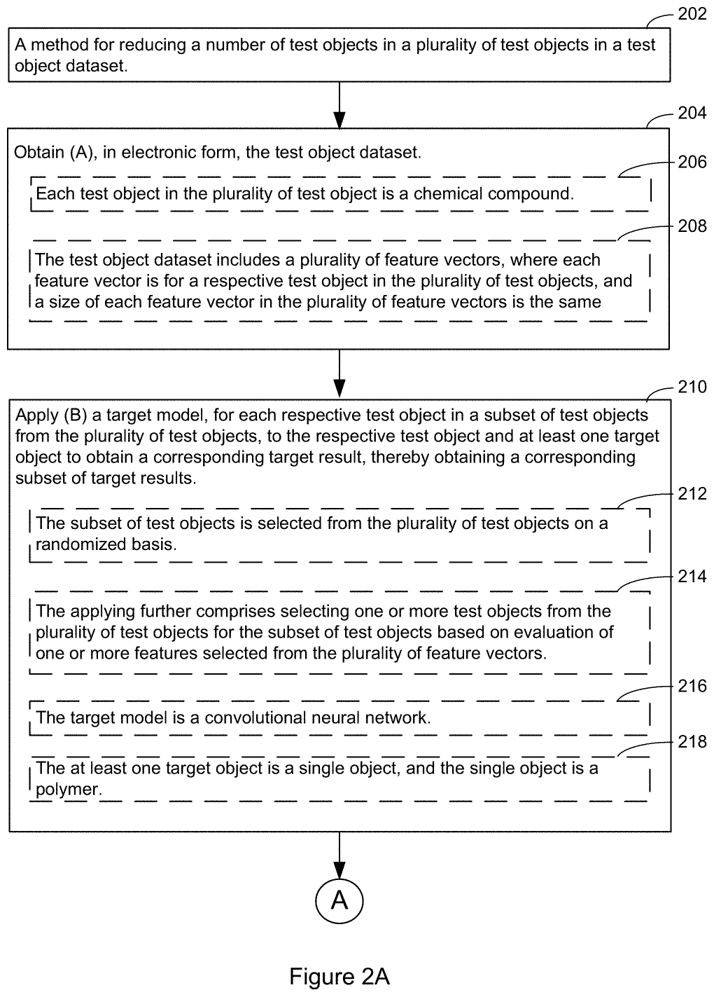

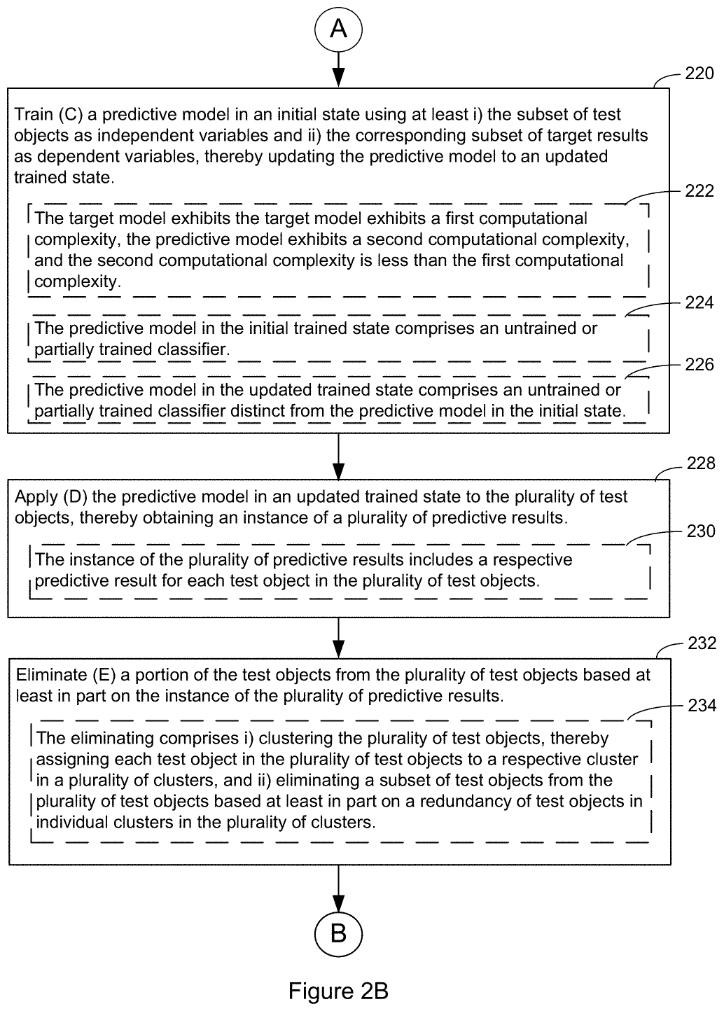

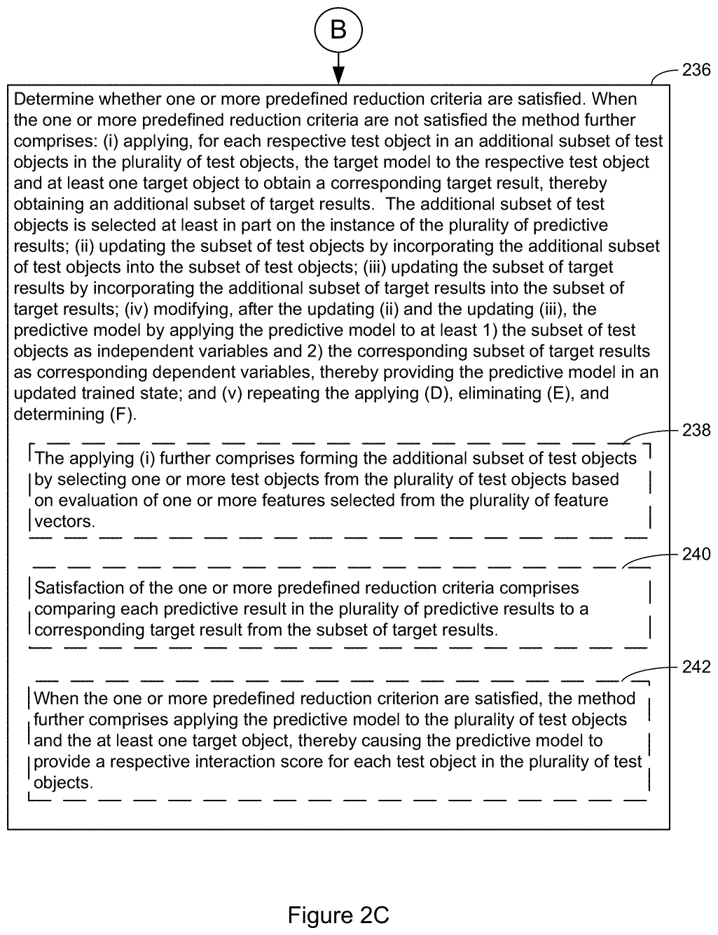

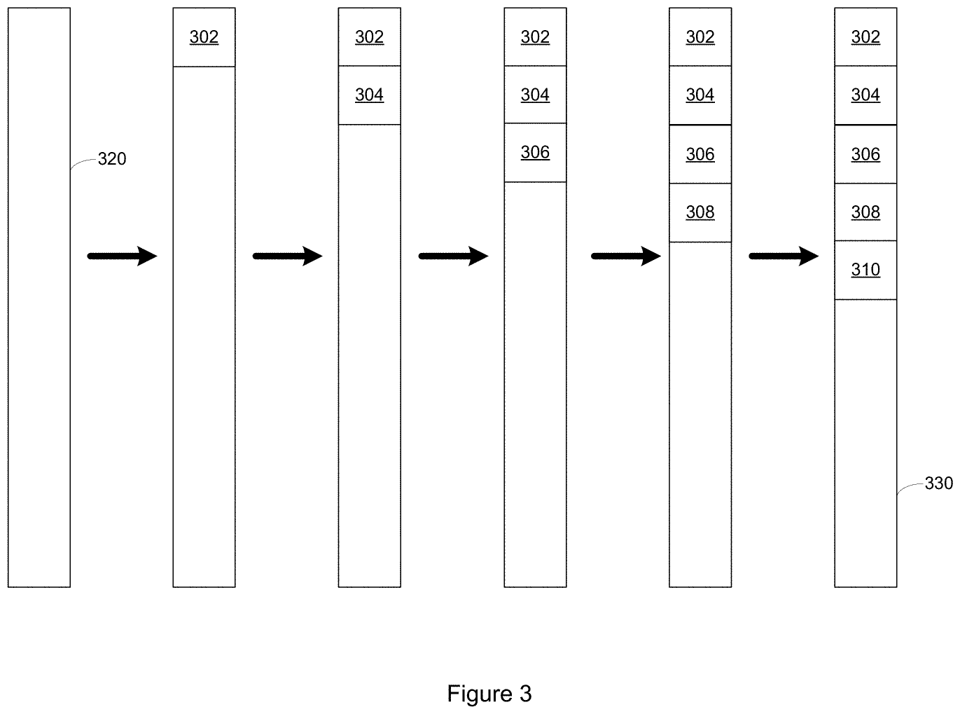

While a system for training a predictive model in accordance with the present disclosure has been disclosed with reference to FIG. 1, methods for performing such training in accordance with the present disclosure are now detailed with reference to FIG. 2 below.

Block 202. Referring to block 202 of FIG. 2A, a method of reducing a number of test objects in a plurality of test objects in a test object dataset is provided.

Blocks 204-206. Referring to block 204 of FIG. 2A, the method proceeds by obtaining, in electronic form, the test object dataset. An example of such a test object dataset is ZINC15. See, Sterling and Irwin, 2005, J. Chem. Inf. Model 45(1), p. 177-182. Zinc 15 is a database of commercially-available compounds for virtual screening. ZINC 15 contains over 230 million purchasable compounds in ready-to-dock, 3D formats. ZINC 15 also contains over 750 million purchasable compounds. Other examples of test object datasets include, but are not limited to MASSIV, AZ Space with Enamine BBs, EVOspace, PGVL, BICLAIM, Lilly, GDB-17, SAVI, CHIPMUNK, REAL ‘Space’, SCUBIDOO 2.1, REAL ‘Database’, WuXi Virtual, PubChem Compounds, Sigma Aldrich ‘in-stock’, eMolecules Plus, and WuXi Chemistry Services, which are summarized in Hoffmann and Gastreich, 2019, “The next level in chemical space navigation: going far beyond enumerable compound libraries,” Drug Discovery Today 24(5), pp. 1148, which is hereby incorporated by reference.

In some embodiments, the plurality of test objects, (e.g., before application of an instance of eliminating a portion of the test objects from the plurality of test objects as described below with regard to blocks 232-234), comprises at least 100 million test objects, at least 500 million test objects, at least 1 billion test objects, at least 2 billion test objects, at least 3 billion test objects, at least 4 billion test objects, at least 5 billion test objects, at least 6 billion test objects, at least 7 billion test objects, at least 8 billion test objects, at least 9 billion test objects, at least 10 billion test objects, at least 11 billion test objects, at least 15 billion test objects, at least 20 billion test objects, at least 30 billion test objects, at least 40 billion test objects, at least 50 billion test objects, at least 60 billion test objects, at least 70 billion test objects, at least 80 billion test objects, at least 90 billion test objects, at least 100 billion test objects, or at least 110 billion test objects. In some embodiments, the plurality of test objects comprises between 100 million and 500 million test objects, between 100 million and 1 billion test objects, between 1 and 2 billion test objects, between 1 and 5 billion test objects, between 1 and 10 billion test objects, between 1 and 15 billion test objects, between 5 and 10 billion test objects, between 5 and 15 billion test objects, or between 10 and 15 billion test objects. In some embodiments, the plurality of test objects is on the order of 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴, 10²⁵, 10²⁶, 10²⁷, 10²⁸, 10²⁹, 10³⁰, 10³¹, 10³², 10³³, 10³⁴, 10³⁵, 10³⁶, 10³⁷, 10³⁸, 10³⁹, 10⁴⁰, 10⁴¹, 10⁴², 10⁴³, 10⁴⁴, 10⁴⁵, 10⁴⁶, 10⁴⁷, 10⁴⁸, 10⁴⁹, 10⁵⁰, 10⁵¹, 10⁵², 10⁵³, 10⁵⁴, 10⁵⁵, 10⁵⁶, 10⁵⁷, 10⁵⁸, 10⁵⁹, or 10⁶⁰ compounds.

In some embodiments, the size of the test object dataset is at least 100 kilobytes, at least 1 megabyte, at least 2 megabytes, at least 3 megabytes, at least 4 megabytes, at least 10 megabytes, at least 20 megabytes, at least 100 megabytes, at least 1 gigabyte, at least 10 gigabytes, or at least 1 terabyte in size. In some embodiments, the test object dataset is a collection of files or datasets (e.g., 2 or more, 3 or more, 4 or more, 100 or more, 1000 or more or one million or more) that collectively have a file size of at least 100 kilobytes, at least 1 megabyte, at least 2 megabytes, at least 3 megabytes, at least 4 megabytes, at least 10 megabytes, at least 20 megabytes, at least 100 megabytes, at least 1 gigabyte, at least 10 gigabytes, or at least 1 terabyte.

With regard to block 206, in some embodiments, each test object in the plurality of test objects represents a respective chemical compound. In some embodiments, each test object represents a chemical compound that satisfies the Lipinski rule of five criterion. In some embodiments, each test object is an organic compounds that satisfies two or more rules, three or more rules, or all four rules of the Lipinski's Rule of Five: (i) not more than five hydrogen bond donors (e.g., OH and NH groups), (ii) not more than ten hydrogen bond acceptors (e.g. N and O), (iii) a molecular weight under 500 Daltons, and (iv) a Log P under 5. The “Rule of Five” is so called because three of the four criteria involve the number five. See, Lipinski, 1997, Adv. Drug Del. Rev. 23, 3, which is hereby incorporated herein by reference in its entirety. In some embodiments, each test object satisfies one or more criteria in addition to Lipinski's Rule of Five. For example, in some embodiments, each test object has five or fewer aromatic rings, four or fewer aromatic rings, three or fewer aromatic rings, or two or fewer aromatic rings. In some embodiments, each test object describes a chemical compound, and the description of the chemical compound comprises modeled atomic coordinates for the chemical compound. In some embodiments, each test object in the plurality of test objects represents a different chemical compound.

In some embodiments, each test object represents an organic compound having a molecular weight of less than 2000 Daltons, of less than 4000 Daltons, of less than 6000 Daltons, of less than 8000 Daltons, of less than 10000 Daltons, or less than 20000 Daltons.

In some embodiments, at least one test object in the plurality of test objects represents a corresponding pharmaceutical compound. In some embodiments, at least one test object in the plurality of test objects represents a corresponding biologically active chemical compound. As used herein, the term “biologically active compound” refers to chemical compounds that have a physiological effect on human beings (e.g., through interactions with proteins). A subset of biologically active chemical compounds can be developed into pharmaceutical drugs. See e.g., Gu et al. 2013 “Use of Natural Products as Chemical Library for Drug Discovery and Network Pharmacology” PLoS One 8(4), e62839. Biologically active compounds can be naturally occurring or synthetic. Various definitions of biological activity have been proposed. See e.g., Lagunin et al. 2000 “PASS: Prediction of activity spectra for biologically active substances” Bioinform 16, 747-748.

In some embodiments, a test object in the test object dataset represents a chemical compound having an “alkyl” group. The term “alkyl” by itself or as part of another substituent of the chemical compound, means, unless otherwise stated, a straight or branched chain, or cyclic hydrocarbon radical, or combination thereof, which may be fully saturated, mono- or polyunsaturated and can include di-, tri- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to optionally include those derivatives of alkyl defined in more detail below, such as “heteroalkyl.” Alkyl groups that are limited to hydrocarbon groups are termed “homoalkyl”. Exemplary alkyl groups include the monounsaturated C₉₋₁₀, oleoyl chain or the diunsaturated C_{9-10, 12-13} linoeyl chain. The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

In some embodiments, a test object in the test object dataset represents a chemical compound having an “alkoxy,” “alkylamino” and “alkylthio” group. The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

In some embodiments, a test object in the test object dataset represents a chemical compound having an “aryloxy” and “heteroaryloxy” group. The terms “aryloxy” and “heteroaryloxy” are used in their conventional sense, and refer to those aryl or heteroaryl groups attached to the remainder of the molecule via an oxygen atom.

In some embodiments, a test object in the test object dataset represents a chemical compound having a “heteroalkyl” group. The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and where the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

In some embodiments, a test object in the test object dataset represents a chemical compound having a “cycloalkyl” and “heterocycloalkyl” group. The terms “cycloalkyl” and “heterocycloalkyl,” by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Further exemplary cycloalkyl groups include steroids, e.g., cholesterol and its derivatives. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

In some embodiments, a test object in the test object dataset represents a chemical compound having a “halo” or “halogen.” The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

In some embodiments, a test object in the test object dataset represents a chemical compound having an “aryl” group. The term “aryl” means, unless otherwise stated, a polyunsaturated, aromatic, substituent that can be a single ring or multiple rings (preferably from 1 to 3 rings), which are fused together or linked covalently.

In some embodiments, a test object in the test object dataset represents a chemical compound having a “heteroaryl” group. The term “heteroaryl” refers to aryl substituent groups (or rings) that contain from one to four heteroatoms selected from N, O, S, Si and B, where the nitrogen and sulfur atoms are optionally oxidized, and the nitrogen atom(s) are optionally quaternized. An exemplary heteroaryl group is a six-membered azine, e.g., pyridinyl, diazinyl and triazinyl. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes aryl, heteroaryl and heteroarene rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl, and “heteroaryl”) are meant to optionally include both substituted and unsubstituted forms of the indicated species. Exemplary substituents for these species are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) of chemical compounds represented by the test object dataset are generically referred to as “alkyl group substituents,” and they can be one or more of a variety of groups selected from, but not limited to: H, substituted or unsubstituted aryl, substituted or unsubstituted heteroaryl, substituted or unsubstituted heterocycloalkyl, —OR′, ═O, ═NR′, ═N—OR′, —NR′R″, SR′, halogen, SiR′R″R′″, OC(O)R′, C(O)R′, CO₂R′, CONR′R″, OC(O)NR′R″, NR″C(O)R′, NR′ C(O)NR″R′″, NR″C(O)2R′, NR C(NR′R″R′″)═NR, NR C(NR′R″)═NR′″, —S(O)R′, —S(O)₂R′, —S(O)₂NR′R″, NRSO2R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like). These terms encompass groups considered exemplary “alkyl group substituents”, which are components of exemplary “substituted alkyl” and “substituted heteroalkyl” moieties.

Similar to the substituents described for the alkyl radical, substituents for the aryl heteroaryl and heteroarene groups are generically referred to as “aryl group substituents.” The substituents are selected from, for example: groups attached to the heteroaryl or heteroarene nucleus through carbon or a heteroatom (e.g., P, N, O, S, Si, or B) including, without limitation, substituted or unsubstituted alkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R″)═NR′″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. Each of the above-named groups is attached to the heteroarene or heteroaryl nucleus directly or through a heteroatom (e.g., P, N, O, S, Si, or B); and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′ and R″″ groups when more than one of these groups is present.

Two of the substituents on adjacent atoms of the aryl, heteroarene or heteroaryl ring may optionally be replaced with a substituent of the formula -T-C(O)—(CRR′)_{q}—U—, where T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ₜ—B—, where A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl, heteroarene or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′ are preferably independently selected from hydrogen or substituted or unsubstituted (C₁-C₆)alkyl. These terms encompass groups considered exemplary “aryl group substituents”, which are components of exemplary “substituted aryl” “substituted heteroarene” and “substituted heteroaryl” moieties.

In some embodiments, a test object in the test object dataset represents a chemical compound having an “acyl” group. As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

In some embodiments, a test object in the test object dataset represents a chemical compound having a “fused ring system”. As used herein, the term “fused ring system” means at least two rings, where each ring has at least 2 atoms in common with another ring. “Fused ring systems” may include aromatic as well as non-aromatic rings. Examples of “fused ring systems” are naphthalenes, indoles, quinolines, chromenes and the like.

As used herein, the term “heteroatom” includes oxygen (O), nitrogen (N), sulfur (S) and silicon (Si), boron (B) and phosphorous (P).

The symbol “R” is a general abbreviation that represents a substituent group that is selected from H, substituted or unsubstituted alkyl, substituted or unsubstituted heteroalkyl, substituted or unsubstituted aryl, substituted or unsubstituted heteroaryl, and substituted or unsubstituted heterocycloalkyl groups.

Block 208. Referring to block 208 of FIG. 2A, in some embodiments, the test object dataset includes a plurality of feature vectors (e.g., where each feature vector corresponds to an individual test object in the test object dataset and includes one or more features). In some embodiments, each respective feature vector in the plurality of feature vectors comprises a chemical fingerprint, molecular fingerprint, one or more computational properties, and/or graph descriptor of the respective chemical compound represented by the corresponding test object. Example molecular fingerprints include, but are not limited to Daylight fingerprints, BCI fingerprints, ECFP fingerprints, ECFC fingerprints, MDL fingerprints, APFP fingerprints, TTFP fingerprints, UNITY 2D fingerprints, and the like.

In some embodiments, some of the features in the vector comprise molecular properties of the corresponding test objects such as any combination of molecular weight, number of rotatable bonds, calculated Log P (e.g., calculated octanol-water partition coefficient or other methods), number of hydrogen-bond donors, number of hydrogen-bond acceptors, number of chiral centers, number of chiral double bonds (E/Z isomerism), polar and apolar desolvation energy (in kcal/mol), net charge, and number of rigid fragments. In some embodiments, one or more test objects in the test object dataset are annotated with function or activity. In some such embodiments the features in the vector comprises such function or activity.

In some embodiments, the test object dataset includes the chemical structure of each test object. For instance, in some embodiments the chemical structure is a SMILES string. In some embodiments, to represent the chemical structure of a test object, a canonical representation of the test object is calculated (e.g., OpenEye's OEchem library, see the Internet at OpenyEye.com). In some embodiments initial 3D models are generated from unambiguous isomeric SMILES of the test object (e.g., using OpenEye's Omega program). In some embodiments, relevant, correctly protonated forms of the test object between pH 5 and 9.5 are then created (e.g., using Schrödinger's ligprep program available from Schrödinger, Inc. on the Internet at schrodinger.com). This includes deprotonating carboxylic acids and tetrazoles and protonating most aliphatic amines, for example. In some embodiments, the partial atomic charges and atomic desolvation penalties for a single 3D conformation of each protonation state, stereoisomer, and tautomer is calculated (e.g., using the semiempirical quantum mechanical program AMSOL16). In some embodiments, OpenEye's program Omega is used to generate 3D conformations. See, for example, Sterling and Irwin, 2005, J. Chem. Inf. Model 45(1), p. 177-182. In some embodiments, the test objects in the test object dataset are represented by the test object dataset, at least in part, with a data structure that is in SMILES, mol2, 3D SDF, DOCK flexibase, or equivalent format.

In embodiments of the test object dataset where test objects are represented by feature vectors, each feature vector is for a respective test object in the plurality of test objects. In some embodiments, a size (e.g., a number of features) of each feature vector in the plurality of feature vectors is the same. In some embodiments, a size (e.g., a number of features) of each feature vector in the plurality of feature vectors is not the same. That is, in some embodiments, at least one of the feature vectors in the plurality of feature vectors is a different size. In some embodiments, each feature vector is an arbitrary length (e.g., each feature vector may be of any size). In some embodiments, the number of dimensions of each feature vector in the plurality of feature vectors may vary (e.g., feature vectors may have any number of dimensions). In some embodiments, each feature vector in the plurality of feature vector is a one-dimensional vector. In some embodiments, one or more feature vectors in the plurality of feature vectors are two-dimensional vectors. In some embodiments, one or more feature vectors in the plurality of feature vectors are three-dimensional vectors. In some embodiments, the number of dimensions of each feature vector in the plurality of feature vectors is the same (e.g., each feature vector has the same number of dimensions). In some embodiments, each feature vector in the plurality of feature vectors is at least a two-dimensional vector. In some embodiments, each feature vector in the plurality of feature vectors is at least an N-dimensional vector, wherein N is a positive integer of two or great (e.g., 2, 3, 4, 5, 6, 7, 8, 9, 10, or greater than 10).

In some embodiments, each respective test object in the plurality of test objects includes a corresponding chemical fingerprint for the chemical compound represented by the respective test object. In some embodiments the chemical fingerprint of a test object is represented by the corresponding feature vector of the test object. As used herein, the term “a chemical fingerprint” refers to a unique pattern (e.g., a unique vector or matrix) corresponding to a particular molecule. In some embodiments, each chemical fingerprint is of a fixed size. In some embodiments, one or more chemical fingerprints are variably sized. In some embodiments, chemical fingerprints for respective test objects in the plurality of test objects can be directly determined (e.g., through mass spectrometry methods such as MALDI-TOF). In some embodiments, chemical fingerprints for respective test objects in the plurality of test objects can be obtained via computational methods. See e.g., Daina et al. (2017) “SwissADME: a free web tool to evaluate pharmacokinetics, drug-likeness and medicinal chemistry friendliness of small molecules” Sci Reports 7, 42717; O'Boyle et al. 2011 “Open Babel: An open chemical toolbox” J Cheminforma 3, 33; Cereto-Massagué et al. 2015 “Molecular fingerprint similarity search in virtual screening” Methods 71, 58-63; and Mitchell 2014 “Machine learning methods in cheminformatics” WIREs Comput Mol Sci. 4:468-481, each of which is hereby incorporated by reference.

Many different methods of representing chemical compounds in computational space are known in the art.

In some embodiments, each chemical fingerprint includes information on an interaction between the respective chemical compound and one or more additional chemical compounds and/or biological macromolecules. In some embodiments, chemical fingerprints comprise information on protein-ligand binding infinity. See Wójcikowski et al. 2018 “Development of a protein-ligand extended connectivity (PLEC) fingerprint and its application for binding affinity predictions” Bioinformatics 35(8), 1334-1341, which is hereby incorporated by reference. In some embodiments, a neural network is used to determine one or more chemical properties (and/or a chemical fingerprint) of at least one test object in the test object database.

In some embodiments, each test object in the test object database corresponds to a known chemical compound with one or more known chemical properties. In some embodiments, the same number of chemical properties are provided for each test object in the plurality of test objects in the test object dataset. In some embodiments, a different number of chemical properties are provided for one or more test objects in the test object dataset. In some embodiments, one or more test objects in the test object dataset are synthetic (e.g., the chemical structure of a test object can be determined despite the fact that the test object has not been analyzed in a lab). See e.g., Gómez-Bombarelli et al. 2017 “Automatic Chemical Design Using a Data-Driven Continuous Representation of Molecules” arXiv:1610.02415v3, which is hereby incorporated by reference.

In some embodiments, graph comparison is used to compare the three-dimensional structure of molecules (e.g., to determine clusters or sets of similar molecules) represented by the test object dataset. The concept of graph comparison relies on comparing graph descriptors and results in dissimilarity or similarity measurements, which can be used for pattern recognition. See e.g., Czech 2011 “Graph Descriptors form B-Matrix Representation” Graph-Based Representations in Patter Recognition, LNCS 6658, 12-21, which is hereby incorporated by reference. In some embodiments, to capture relevant structural properties within a graph (e.g., of sets of test objects), measurements such as clustering coefficient, efficiency, or betweenness centrality can be utilized. See e.g. Costa et al. 2007 “Characterization of complex networks: A survey of measurements” Advances Phys 56(1), 198-200, which is hereby incorporated by reference.

Block 210. Referring to block 210 of FIG. 2A, for each respective test object in a subset of test objects from the plurality of test objects, a target model is applied to the respective test object and at least one target object to obtain a corresponding target result, thereby obtaining a corresponding subset of target results. In typical embodiments the respective test object is docked to each target object of the at least one target object. In some embodiments there is only a single target object.

In some embodiments, a target object is a polymer. Examples of polymers include, but are not limited to proteins, polypeptides, polynucleic acids, polyribonucleic acids, polysaccharides, or assemblies of any combination thereof. A polymer, such as those studied using some embodiments of the disclosed systems and methods, is a large molecule composed of repeating residues. In some embodiments, the polymer is a natural material. In some embodiments, the polymer is a synthetic material. In some embodiments, the polymer is an elastomer, shellac, amber, natural or synthetic rubber, cellulose, Bakelite, nylon, polystyrene, polyethylene, polypropylene, polyacrylonitrile, polyethylene glycol, or a polysaccharide.

In some embodiments, a target object is a heteropolymer (copolymer). A copolymer is a polymer derived from two (or more) monomeric species, as opposed to a homopolymer where only one monomer is used. Copolymerization refers to methods used to chemically synthesize a copolymer. Examples of copolymers include, but are not limited to, ABS plastic, SBR, nitrile rubber, styrene-acrylonitrile, styrene-isoprene-styrene (SIS) and ethylene-vinyl acetate. Since a copolymer consists of at least two types of constituent units (also structural units, or particles), copolymers can be classified based on how these units are arranged along the chain. These include alternating copolymers with regular alternating A and B units. See, for example, Jenkins, 1996, “Glossary of Basic Terms in Polymer Science,” Pure Appl. Chem. 68 (12): 2287-2311, which is hereby incorporated herein by reference in its entirety. Additional examples of copolymers are periodic copolymers with A and B units arranged in a repeating sequence (e.g. (A-B-A-B-B-A-A-A-A-B-B-B)n). Additional examples of copolymers are statistical copolymers in which the sequence of monomer residues in the copolymer follows a statistical rule. See, for example, Painter, 1997, Fundamentals of Polymer Science, CRC Press, 1997, p 14, which is hereby incorporated by reference herein in its entirety. Still other examples of copolymers that may be evaluated using the disclosed systems and methods are block copolymers comprising two or more homopolymer subunits linked by covalent bonds. The union of the homopolymer subunits may require an intermediate non-repeating subunit, known as a junction block. Block copolymers with two or three distinct blocks are called diblock copolymers and triblock copolymers, respectively.

In some embodiments, a target object is in fact a plurality of polymers, where the respective polymers in the plurality of polymers do not all have the same molecular weight. In some such embodiments, the polymers in the plurality of polymers fall into a weight range with a corresponding distribution of chain lengths. In some embodiments, the polymer is a branched polymer molecule comprising a main chain with one or more substituent side chains or branches. Types of branched polymers include, but are not limited to, star polymers, comb polymers, brush polymers, dendronized polymers, ladders, and dendrimers. See, for example, Rubinstein et al., 2003, Polymer physics, Oxford; New York: Oxford University Press. p. 6, which is hereby incorporated by reference herein in its entirety.

In some embodiments, a target object is a polypeptide. As used herein, the term “polypeptide” means two or more amino acids or residues linked by a peptide bond. The terms “polypeptide” and “protein” are used interchangeably herein and include oligopeptides and peptides. An “amino acid,” “residue” or “peptide” refers to any of the twenty standard structural units of proteins as known in the art, which include imino acids, such as proline and hydroxyproline. The designation of an amino acid isomer may include D, L, R and S. The definition of amino acid includes nonnatural amino acids. Thus, selenocysteine, pyrrolysine, lanthionine, 2-aminoisobutyric acid, gamma-aminobutyric acid, dehydroalanine, ornithine, citrulline and homocysteine are all considered amino acids. Other variants or analogs of the amino acids are known in the art. Thus, a polypeptide may include synthetic peptidomimetic structures such as peptoids. See Simon et al., 1992, Proceedings of the National Academy of Sciences USA, 89, 9367, which is hereby incorporated by reference herein in its entirety. See also Chin et al., 2003, Science 301, 964; and Chin et al., 2003, Chemistry & Biology 10, 511, each of which is incorporated by reference herein in its entirety.

In some embodiments, a target object evaluated in accordance with some embodiments of the disclosed systems and methods may also have any number of posttranslational modifications. Thus, a target object may include those polymers that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphotases and kinases). Other types of posttranslational modifications are known in the art and are also included.

In some embodiments, a target object is an organometallic complex. An organometallic complex is chemical compound containing bonds between carbon and metal. In some instances, organometallic compounds are distinguished by the prefix “organo-” e.g. organopalladium compounds.

In some embodiments, a target object is a surfactant. Surfactants are compounds that lower the surface tension of a liquid, the interfacial tension between two liquids, or that between a liquid and a solid. Surfactants may act as detergents, wetting agents, emulsifiers, foaming agents, and dispersants. Surfactants are usually organic compounds that are amphiphilic, meaning they contain both hydrophobic groups (their tails) and hydrophilic groups (their heads). Therefore, a surfactant molecule contains both a water insoluble (or oil soluble) component and a water soluble component. Surfactant molecules will diffuse in water and adsorb at interfaces between air and water or at the interface between oil and water, in the case where water is mixed with oil. The insoluble hydrophobic group may extend out of the bulk water phase, into the air or into the oil phase, while the water soluble head group remains in the water phase. This alignment of surfactant molecules at the surface modifies the surface properties of water at the water/air or water/oil interface.

Examples of ionic surfactants include ionic surfactants such as anionic, cationic, or zwitterionic (ampoteric) surfactants. In some embodiments, the target object is a reverse micelle or liposome.

In some embodiments, a target object is a fullerene. A fullerene is any molecule composed entirely of carbon, in the form of a hollow sphere, ellipsoid or tube. Spherical fullerenes are also called buckyballs, and they resemble the balls used in association football. Cylindrical ones are called carbon nanotubes or buckytubes. Fullerenes are similar in structure to graphite, which is composed of stacked graphene sheets of linked hexagonal rings; but they may also contain pentagonal (or sometimes heptagonal) rings.

In some embodiments, a target object is a polymer and the spatial coordinates are a set of three-dimensional coordinates {x₁, . . . , x_{N}} for a crystal structure of the polymer resolved at a resolution of 2.5 Å or better (208), where N is an integer of two or greater (e.g., 10 or greater, 20 or greater, etc.). In some embodiments, the target object is a polymer and the spatial coordinates are a set of three-dimensional coordinates {x1, . . . , xN} for a crystal structure of the polymer resolved at a resolution of 3.3 Å or better (210). In some embodiments, the target object is a polymer and the spatial coordinates are a set of three-dimensional coordinates {x₁, . . . , x_{N}} for a crystal structure of the polymer resolved (e.g., by X-ray crystallographic techniques) at a resolution of 3.3 Å or better, 3.2 Å or better, 3.1 Å or better, 3.0 Å or better, 2.5 Å or better, 2.2 Å or better, 2.0 Å or better, 1.9 Å or better, 1.85 Å or better, 1.80 Å or better, 1.75 Å or better, or 1.70 Å or better.

In some embodiments, a target object is a polymer and the spatial coordinates are an ensemble of ten or more, twenty or more or thirty or more three-dimensional coordinates for the polymer determined by nuclear magnetic resonance where the ensemble has a backbone RMSD of 1.0 Å or better, 0.9 Å or better, 0.8 Å or better, 0.7 Å or better, 0.6 Å or better, 0.5 Å or better, 0.4 Å or better, 0.3 Å or better, or 0.2 Å or better. In some embodiments the spatial coordinates are determined by neutron diffraction or cryo-electron microscopy.

In some embodiments, a target object includes two different types of polymers, such as a nucleic acid bound to a polypeptide. In some embodiments, the native polymer includes two polypeptides bound to each other. In some embodiments, the native polymer under study includes one or more metal ions (e.g. a metalloproteinase with one or more zinc atoms). In such instances, the metal ions and or the organic small molecules may be included in the spatial coordinates for the target object.

In some embodiments the target object is a polymer and there are ten or more, twenty or more, thirty or more, fifty or more, one hundred or more, between one hundred and one thousand, or less than 500 residues in the polymer.

In some embodiments, the spatial coordinates of the target object are determined using modeling methods such as ab initio methods, density functional methods, semi-empirical and empirical methods, molecular mechanics, chemical dynamics, or molecular dynamics.

In an embodiment, the spatial coordinates are represented by the Cartesian coordinates of the centers of the atoms comprising the target object. In some alternative embodiments, the spatial coordinates for a target object are represented by the electron density of the target object as measured, for example, by X-ray crystallography. For example, in some embodiments, the spatial coordinates comprise a 2F_{observed}−F_{calculated} electron density map computed using the calculated atomic coordinates of the target object, where F_{observed} is the observed structure factor amplitudes of the target object and Fc is the structure factor amplitudes calculated from the calculated atomic coordinates of a target object.

Thus spatial coordinates for a target object may be received as input data from a variety of sources, such as, but not limited to, structure ensembles generated by solution NMR, co-complexes as interpreted from X-ray crystallography, neutron diffraction, or cryo-electron microscopy, sampling from computational simulations, homology modeling or rotamer library sampling, and combinations of these techniques.

In some embodiments, block 210 encompasses obtaining spatial coordinates for the target object. Further, block 210 encompasses modeling the respective test object with the target object in each pose of a plurality of different poses, thereby creating a plurality of voxel maps, where each respective voxel map in the plurality of voxel maps comprises the respective test object in a respective pose in the plurality of different poses.

In some embodiments, a target object is a polymer with an active site, the respective test object is a chemical compound, and the modeling the respective test object with the target object in each pose in a plurality of different poses comprises docking the test object into the active site of the target object. In some embodiments, the respective test object is docked onto the target object a plurality of times to form the plurality of poses (e.g. each docking representing a different pose). In some embodiments, the test object is docked onto the target object twice, three times, four times, five or more times, ten or more times, fifty or more times, 100 or more times, or a 1000 or more times. Each such docking represents a different pose of the respective test object docked onto the target object. In some embodiments, the respective target object is a polymer with an active site and the test object is docked into the active site in each of plurality of different ways, each such way representing a different pose. It is expected that many of these poses are not correct, meaning that such poses do not represent true interactions between the respective test object and the target object that arise in nature. Without intending to be limited by any particular theory, it is expected that inter-object (e.g., intermolecular) interactions observed among incorrect poses will cancel each other out like white noise whereas the inter-object interactions formed by correct poses formed by test objects will reinforce each other. In some embodiments, test objects are docked by either random pose generation techniques, or by biased pose generation. In some embodiments, test objects are docked by Markov chain Monte Carlo sampling. In some embodiments, such sampling allows the full flexibility of test objects in the docking calculations and a scoring function that is the sum of the interaction energy between the test object and the target object as well as the conformational energy of the test object. See, for example, Liu and Wang, 1999, “MCDOCK: A Monte Carlo simulation approach to the molecular docking problem,” Journal of Computer-Aided Molecular Design 13, 435-451, which is hereby incorporated by reference.

In some embodiments, algorithms such as DOCK (Shoichet, Bodian, and Kuntz, 1992, “Molecular docking using shape descriptors,” Journal of Computational Chemistry 13(3), pp. 380-397; and Knegtel, Kuntz, and Oshiro, 1997 “Molecular docking to ensembles of protein structures,” Journal of Molecular Biology 266, pp. 424-440, each of which is hereby incorporated by reference) are used to find a plurality of poses for each respective test object against each of the target objects. Such algorithms model the target object and the test object as rigid bodies. The docked conformation is searched using surface complementary to find poses.

In some embodiments, algorithms such as AutoDOCK (Morris et al., 2009, “AutoDock4 and AutoDockTools4: Automated Docking with Selective Receptor Flexibility,” J. Comput. Chem. 30(16), pp. 2785-2791; Sotriffer et al., 2000, “Automated docking of ligands to antibodies: methods and applications,” Methods: A Companion to Methods in Enzymology 20, pp. 280-291; and “Morris et al., 1998, “Automated Docking Using a Lamarckian Genetic Algorithm and Empirical Binding Free Energy Function,” Journal of Computational Chemistry 19: pp. 1639-1662, each of which is hereby incorporated by reference) are used to find a plurality of poses for each respective test object against each of the target objects. AutoDOCK uses a kinematic model of the ligand and supports Monte Carlo, simulated annealing, the Lamarckian Genetic Algorithm, and Genetic algorithms. Accordingly, in some embodiments the plurality of different poses (for a given test object-target object pair) are obtained by Markov chain Monte Carlo sampling, simulated annealing, Lamarckian Genetic Algorithms, or genetic algorithms, using a docking scoring function.

In some embodiments, algorithms such as FlexX (Rarey et al., 1996, “A Fast Flexible Docking Method Using an Incremental Construction Algorithm,” Journal of Molecular Biology 261, pp. 470-489, which is hereby incorporated by reference) are used to find a plurality of poses for each of the respective test objects in the subset of test object against each of the target objects. FlexX does an incremental construction of a test object at the active site of a target object using a greedy algorithm. Accordingly, in some embodiments the plurality of different poses (for a given test object-target object pair) are obtained by a greedy algorithm.

In some embodiments, algorithms such as GOLD (Jones et al., 1997, “Development and Validation of a Genetic Algorithm for flexible Docking,” Journal Molecular Biology 267, pp. 727-748, which is hereby incorporated by reference) are used to find a plurality of poses for each of the test objects in the subset of test objects against each of the target objects. GOLD stands for Genetic Optimization for Ligand Docking. GOLD builds a genetically optimized hydrogen bonding network between the test object and the target object.

In some embodiments, the modeling comprises performing a molecular dynamics run of the target object and the test object. During the molecular dynamics run, the atoms of the target object and the test object are allowed to interact for a fixed period of time, giving a view of the dynamical evolution of the system. The trajectory of atoms in the target object and the test object are determined by numerically solving Newton's equations of motion for a system of interacting particles, where forces between the particles and their potential energies are calculated using interatomic potentials or molecular mechanics force fields. See, Alder and Wainwright, 1959, “Studies in Molecular Dynamics. I. General Method,”. J. Chem. Phys. 31 (2): 459; and Bibcode, 1959, J.Ch.Ph. 31, 459A, doi:10.1063/1.1730376, each of which is hereby incorporated by reference. Thus, in this way, the molecular dynamics run produces a trajectory of the target object and the test object together over time. This trajectory comprises the trajectory of the atoms in the target object and the test object. In some embodiments, a subset of the plurality of different poses is obtained by taking snapshots of this trajectory over a period of time. In some embodiments, poses are obtained from snapshots of several different trajectories, where each trajectory comprise a different molecular dynamics run of the target object interacting with the test object. In some embodiments, prior to a molecular dynamics run, a test object is first docked into an active site of the target object using a docking technique.

Regardless of what modeling method is used, what is achieved for any given test object−target object pair is a diverse set of poses of the test object with the target object with the expectation that one or more of the poses is close enough to the naturally occurring pose to demonstrate some of the relevant intermolecular interactions between the given test object/target object pair.

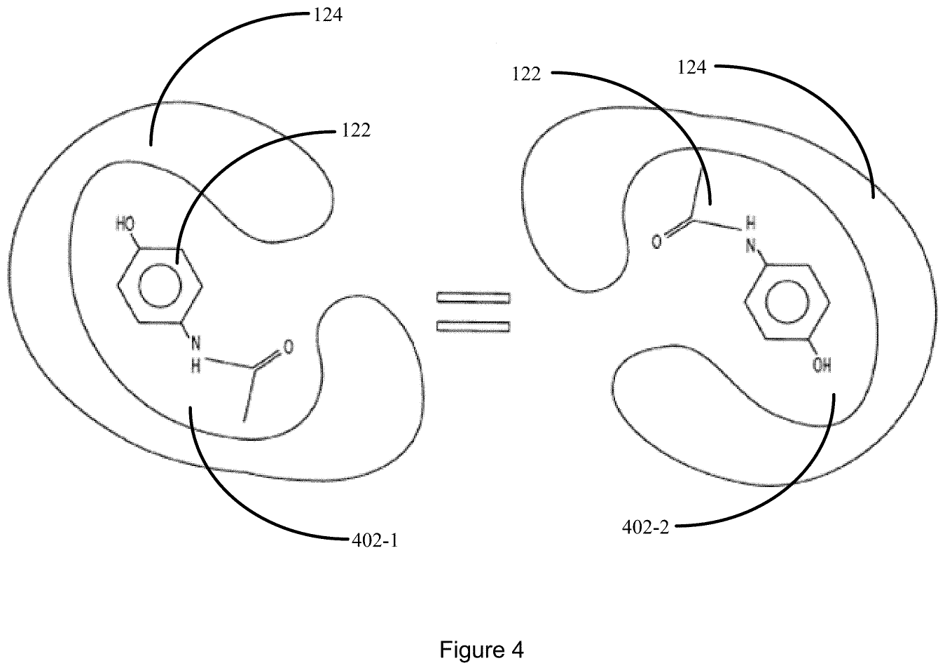

In some embodiments an initial pose of the test object in the active site of a target object is generated using any of the above-described techniques and additional poses are generated through the application of some combination of rotation, translation, and mirroring operators in any combination of the three X, Y and Z planes. Rotation and translation of the test may be randomly selected (within some range, e.g. plus or minus 5 Å from the origin) or uniformly generated at some pre-specified increment (e.g., all 5 degree increments around the circle). FIG. 4 provides a sample illustration of a test object 122 in two different poses (402-1 and 402-2) in the active site of a target object 124.

After generation of each of the poses for each of the target and/or test objects, in some embodiments a voxel map is created of each pose thereby creating a plurality of voxel maps for a given respective target object with respect to a target object. In some embodiments, each respective voxel map in the plurality of voxel maps is created by a method comprising: (i) sampling the test object, in a respective pose in the plurality of different poses, and the target object on a three-dimensional grid basis thereby forming a corresponding three dimensional uniform space-filling honeycomb comprising a corresponding plurality of space filling (three-dimensional) polyhedral cells and (ii) populating, for each respective three-dimensional polyhedral cell in the corresponding plurality of three-dimensional cells, a voxel (discrete set of regularly-spaced polyhedral cells) in the respective voxel map based upon a property (e.g., chemical property) of the respective three-dimensional polyhedral cell. Thus, if a particular test object has ten poses relative to a target object, ten corresponding voxel maps are created, if a particular test object has one hundred poses relative to a target object, one hundred corresponding voxel maps are created, and so forth in such embodiments. Examples of space filling honeycombs include cubic honeycombs with parallelepiped cells, hexagonal prismatic honeycombs with hexagonal prism cells, rhombic dodecahedra with rhombic dodecahedron cells, elongated dodecahedra with elongated dodecahedron cells, and truncated octahedra with truncated octahedron cells.

In some embodiments, the space filling honeycomb is a cubic honeycomb with cubic cells and the dimensions of such voxels determine their resolution. For example, a resolution of 1 Å may be chosen meaning that each voxel, in such embodiments, represents a corresponding cube of the geometric data with 1 Å dimensions (e.g., 1 Å×1 Å×1 Å in the respective height, width, and depth of the respective cells). However, in some embodiments, finer grid spacing (e.g., 0.1 Å or even 0.01 Å) or coarser grid spacing (e.g. 4 Å) is used, where the spacing yields an integer number of voxels to cover the input geometric data. In some embodiments, the sampling occurs at a resolution that is between 0.1 Å and 10 Å. As an illustration, for a 40 Å input cube, with a 1 Å resolution, such an arrangement would yield 40*40*40=64,000 input voxels.

In some embodiments, the respective test object is a first compound and the target object is a second compound, a characteristic of an atom incurred in the sampling (i) is placed in a single voxel in the respective voxel map by the populating (ii), and each voxel in the plurality of voxels represents a characteristic of a maximum of one atom. In some embodiments, the characteristic of the atom consists of an enumeration of the atom type. As one example, for biological data, some embodiments of the disclosed systems and methods are configured to represent the presence of every atom in a given voxel of the voxel map as a different number for that entry, e.g., if a carbon is in a voxel, a value of 6 is assigned to that voxel because the atomic number of carbon is 6. However, such an encoding could imply that atoms with close atomic numbers will behave similarly, which may not be particularly useful depending on the application. Further, element behavior may be more similar within groups (columns on the periodic table), and therefore such an encoding poses additional work for the convolutional neural network to decode.

In some embodiments, the characteristic of the atom is encoded in the voxel as a binary categorical variable. In such embodiments, atom types are encoded in what is termed a “one-hot” encoding: every atom type has a separate channel. Thus, in such embodiments, each voxel has a plurality of channels and at least a subset of the plurality of channels represent atom types. For example, one channel within each voxel may represent carbon whereas another channel within each voxel may represent oxygen. When a given atom type is found in the three-dimensional grid element corresponding to a given voxel, the channel for that atom type within the given voxel is assigned a first value of the binary categorical variable, such as “1”, and when the atom type is not found in the three-dimensional grid element corresponding to the given voxel, the channel for that atom type is assigned a second value of the binary categorical variable, such as “0” within the given voxel.

While there are over 100 elements, most are not encountered in biology. However, even representing the most common biological elements (e.g., H, C, N, O, F, P, S, Cl, Br, I, Li, Na, Mg, K, Ca, Mn, Fe, Co, Zn) may yield 18 channels per voxel or 10,483*18=188,694 inputs to the receptor field. As such, in some embodiments, each respective voxel in a voxel map in the plurality of voxel maps comprises a plurality of channels, and each channel in the plurality of channels represents a different property that may arise in the three-dimensional space filling polyhedral cell corresponding to the respective voxel. The number of possible channels for a given voxel is even higher in those embodiments where additional characteristics of the atoms (for example, partial charge, presence in ligand versus protein target, electronegativity, or SYBYL atom type) are additionally presented as independent channels for each voxel, necessitating more input channels to differentiate between otherwise-equivalent atoms.

In some embodiments, each voxel has five or more input channels. In some embodiments, each voxel has fifteen or more input channels. In some embodiments, each voxel has twenty or more input channels, twenty-five or more input channels, thirty or more input channels, fifty or more input channels, or one hundred or more input channels. In some embodiments, each voxel has five or more input channels selected from the descriptors found in Table 1 below. For example, in some embodiments, each voxel has five or more channels, each encoded as a binary categorical variable where each such channel represents a SYBYL atom type selected from Table 1 below. For instance, in some embodiments, each respective voxel in a voxel map includes a channel for the C.3 (sp3 carbon) atom type meaning that if the grid in space for a given test object-target object complex represented by the respective voxel encompasses an sp3 carbon, the channel adopts a first value (e.g., “1”) and is a second value (e.g. “0”) otherwise.

TABLE 1
SYBYL Atom Types

| SYBYL ATOM TYPE | DESCRIPTION |
|---|---|
| C.3 | sp3 carbon |
| C.2 | sp2 carbon |
| C.ar | aromatic carbon |
| C.1 | sp carbon |
| N.3 | sp3 nitrogen |
| N.2 | sp2 nitrogen |
| N.1 | sp nitrogen |
| O.3 | sp3 oxygen |
| O.2 | sp2 oxygen |
| S.3 | sp3 sulfur |
| N.ar | aromatic nitrogen |
| P.3 | sp3 phosphorous |
| H | hydrogen |
| Br | bromine |
| Cl | chlorine |
| F | fluorine |
| I | iodine |
| S.2 | sp2 sulfur |
| N.pl3 | pl3 trigonal planar nitrogen |
| LP | lone pair |
| Na | sodium |
| K | potassium |
| Ca | calcium |
| Li | lithium |
| Al aluminum | aluminum |
| Si | silicon |
| N.am | amide nitrogen |
| S.o | sulfoxide sulfur |
| S.o2 | sulfone sulfur |
| N.4 | positively charged nitrogen |
| O.co2 | oxygen in carboxylate or phosphate group |
| C.cat | carbocation, used only in a guadinium group |
| H.spc | hydrogen in SPC water model |
| O.spc | oxygen in SPC water model |
| H.t3p | hydrogen in TIP3P water model |
| O.t3p | oxygen in TIP3P water model |
| ANY | any atom |
| HEV | heavy (non H) atom |
| HET | heteroatom (N, O, S, P) |
| HAL | halogen |
| Mg | magnesium |
| Cr.oh | hydroxy chromium |
| Cr.th | chromium |
| Se | selenium |
| Fe | iron |
| Cu | copper |
| Zn | zinc |
| Sn | tin |
| Mo | molybdenum |
| Mn | manganese |
| Co.oh | hydroxy cobalt |

In some embodiments, each voxel comprises ten or more input channels, fifteen or more input channels, or twenty or more input channels selected from the descriptors found in Table 1 above. In some embodiments, each voxel includes a channel for halogens.

In some embodiments, a structural protein-ligand interaction fingerprint (SPLIF) score is generated for each pose of a respective test object to a target object and this SPLIF score is used as additional input into the target model or is individually encoded in the voxel map. For a description of SPLIFs, see Da and Kireev, 2014, J. Chem. Inf. Model. 54, pp. 2555-2561, “Structural Protein—Ligand Interaction Fingerprints (SPLIF) for Structure-Based Virtual Screening: Method and Benchmark Study,” which is hereby incorporated by reference. A SPLIF implicitly encodes all possible interaction types that may occur between interacting fragments of the test object and the target object (e.g., π-π, CH-π, etc.). In the first step, a test object-target object complex (pose) is inspected for intermolecular contacts. Two atoms are deemed to be in a contact if the distance between them is within a specified threshold (e.g., within 4.5 Å). For each such intermolecular atom pair, the respective test atom and target object atoms are expanded to circular fragments, e.g., fragments that include the atoms in question and their successive neighborhoods up to a certain distance. Each type of circular fragment is assigned an identifier. In some embodiments, such identifiers are coded in individual channels in the respective voxels. In some embodiments, the Extended Connectivity Fingerprints up to the first closest neighbor (ECFP2) as defined in the Pipeline Pilot software can be used. See, Pipeline Pilot, ver. 8.5, Accelrys Software Inc., 2009, which is hereby incorporated by reference. ECFP retains information about all atom/bond types and uses one unique integer identifier to represent one substructure (e.g., circular fragment). The SPLIF fingerprint encodes all the circular fragment identifiers found. In some embodiments, the SPLIF fingerprint is not encoded individual voxels but serves as a separate independent input in the target model.

In some embodiments, rather than or in addition to SPLIFs, structural interaction fingerprints (SIFt) are computed for each pose of a given test object to a target object and independently provided as input into the target model or are encoded in the voxel map. For a computation of SIFts, see Deng et al., 2003, “Structural Interaction Fingerprint (SIFt): A Novel Method for Analyzing Three-Dimensional Protein-Ligand Binding Interactions,” J. Med. Chem. 47 (2), pp. 337-344, which is hereby incorporated by reference.

In some embodiments, rather than or in addition to SPLIFs and SIFTs, atom-pairs-based interaction fragments (APIFs) are computed for each pose of a given test object to a target object and independently provided as input into the target model or is individually encoded in the voxel map. For a computation of APIFs, see Perez-Nueno et al., 2009, “APIF: a new interaction fingerprint based on atom pairs and its application to virtual screening,” J. Chem. Inf. Model. 49(5), pp. 1245-1260, which is hereby incorporated by reference.

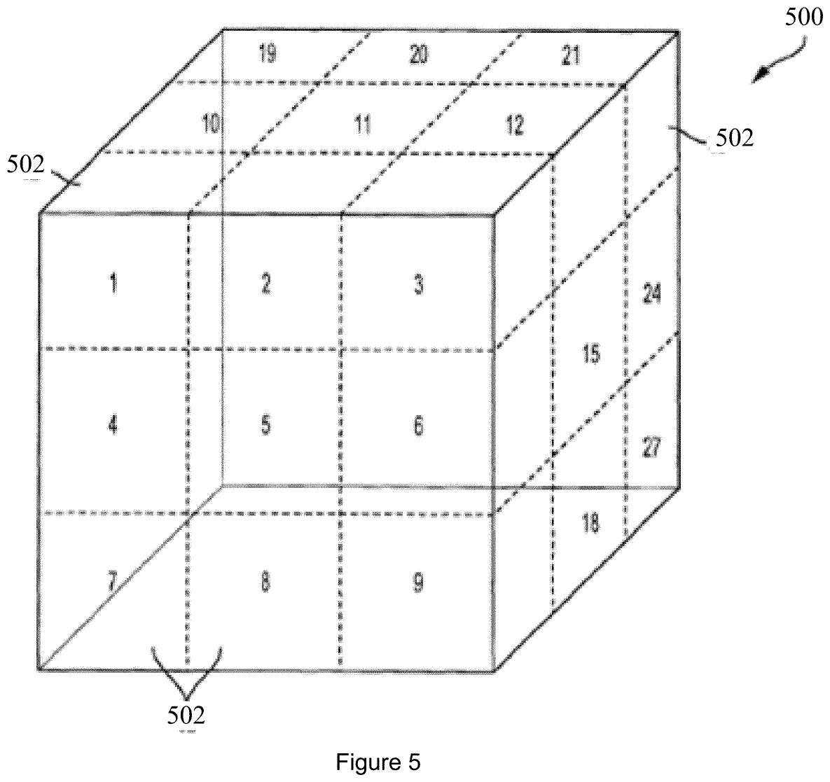

The data representation may be encoded with the biological data in a way that enables the expression of various structural relationships associated with molecules/proteins for example. The geometric representation may be implemented in a variety of ways and topographies, according to various embodiments. The geometric representation is used for the visualization and analysis of data. For example, in an embodiment, geometries may be represented using voxels laid out on various topographies, such as 2-D, 3-D Cartesian/Euclidean space, 3-D non-Euclidean space, manifolds, etc. For example, FIG. 5 illustrates a sample three-dimensional grid structure 500 including a series of sub-containers, according to an embodiment. Each sub-container 502 may correspond to a voxel. A coordinate system may be defined for the grid, such that each sub-container has an identifier. In some embodiments of the disclosed systems and methods, the coordinate system is a Cartesian system in 3-D space, but in other embodiments of the system, the coordinate system may be any other type of coordinate system, such as a oblate spheroid, cylindrical or spherical coordinate systems, polar coordinates systems, other coordinate systems designed for various manifolds and vector spaces, among others. In some embodiments, the voxels may have particular values associated to them, which may, for example, be represented by applying labels, and/or determining their positioning, among others.

In some embodiments, block 210 further comprises unfolding each voxel map in the plurality of voxel maps into a corresponding vector, thereby creating a plurality of vectors, where each vector in the plurality of vectors is the same size. In some embodiments, each respective vector in the plurality of vectors is inputted into the target model. In some embodiments the target model includes (i) an input layer for sequentially receiving the plurality of vectors, (ii) a plurality of convolutional layers, and (iii) a scorer, where the plurality of convolutional layers includes an initial convolutional layer and a final convolutional layer, and each layer in the plurality of convolutional layers is associated with a different set of weights. In such embodiments, responsive to input of a respective vector in the plurality of vectors, the input layer feeds a first plurality of values into the initial convolutional layer as a first function of values in the respective vector, each respective convolutional layer, other than the final convolutional layer, feeds intermediate values, as a respective second function of (i) the different set of weights associated with the respective convolutional layer and (ii) input values received by the respective convolutional layer, into another convolutional layer in the plurality of convolutional layers, and the final convolutional layer feeds final values, as a third function of (i) the different set of weights associated with the final convolutional layer and (ii) input values received by the final convolutional layer, into the scorer. In this way, a plurality of scores are obtained from the scorer, where each score in the plurality of scores corresponds to the input of a vector in the plurality of vectors into the input layer. The plurality of scores are then used to provide the corresponding target result for the respective test object. In some embodiments, the target result is a weighted mean of the plurality of scores. In some embodiments, the target result is a measure of central tendency of the plurality of scores. Examples of a measure of central tendency include the arithmetic mean, weighted mean, midrange, midhinge, trimean, Winsorized mean, median, or mode of the plurality of scores.

In some embodiments, the scorer comprises a plurality of fully-connected layers and an evaluation layer where a fully-connected layer in the plurality of fully-connected layers feeds into the evaluation layer. In some embodiments, the scorer comprises a decision tree, a multiple additive regression tree, a clustering algorithm, principal component analysis, a nearest neighbor analysis, a linear discriminant analysis, a quadratic discriminant analysis, a support vector machine, an evolutionary method, a projection pursuit, and ensembles thereof. In some embodiments, each vector in the plurality of vectors is a one-dimensional vector. In some embodiments, the plurality of different poses comprises 2 or more poses, 10 or more poses, 100 or more poses, or 1000 or more poses. In some embodiments, the plurality of different poses is obtained using a docking scoring function in one of markup chain Monte Carlo sampling, simulated annealing, Lamarckian Genetic Algorithms, or genetic algorithms. In some embodiments, the plurality of different poses is obtained by incremental search using a greedy algorithm.

Blocks 212 and 214. In some embodiments, the target model has a higher computational complexity than the predictive model. In some such embodiments it is computationally prohibitive to apply the target model to every test object in the test object dataset. For this reason, the target model is typically applied to a subset of test objects rather than every test object in the test object dataset. In some embodiments, some level of diversity in the subset of test objects (e.g., the subset of test objects comprising test objects with a range of structural or functional qualities) is desired. In some embodiments, the subset of test objects comprises at least 1,000 test objects, at least 5,000 test objects, at least 10,000 test objects, at least 25,000 test objects, at least 50,000 test objects, at least 75,000 test objects, at least 100,000 test objects, at least 250,000 test objects, at least 500,000 test objects, at least 750,000 test objects, at least 1 million test objects, at least 2 million test objects, at least 3 million test objects, at least 4 million test objects, at least 5 million test objects, at least 6 million test objects, at least 7 million test objects, at least 8 million test objects, at least 9 million test objects, or at least 10 million test objects.

To ensure this, referring to block 212 of FIG. 2A, in some embodiments the subset of test objects is selected from the test object dataset on a randomized basis (e.g., the subset of test objects is selected from the test object dataset using any random method known in the art).

Referring to block 214 of FIG. 2A, in other embodiments, the subset of test objects is selected from the test object dataset based on an evaluation of one or more features of the feature vectors of the test objects. In some such embodiments, evaluation of features comprises making a selection of test objects from the plurality of test objects based on clustering (e.g., selecting test objects from multiple clusters when forming each subset of test objects). Then, the subset of test objects is selected based at least in part on a redundancy of test objects in individual clusters in the plurality of clusters (e.g., to obtain a subset of test objects that are representative of different types of chemical compounds). For example, consider the case in which the test objects of the test object dataset are clustered, based on their feature vectors, into 100 different clusters. One approach to selecting the subset of test objects is to select a fixed number of test objects (e.g., 10, 100, 1000, etc.) from each of the different clusters in order to form the subset of test objects. Within each cluster, the selection of test objects can be on a random basis. Alternatively, within each cluster, those test objects that are closest to the center of each cluster are selected on the basis that such test objects most represent the properties of their respective clusters. In some embodiments, the form of clustering that is used is unsupervised clustering. A benefit of clustering the plurality of test objects from the test object dataset is that this provides for more accurate training of the predictive model. If, for example, all or the majority of the test objects in a subset of test objects are similar chemical compounds (e.g., including a same chemical group, having a similar structure, etc.), there is a risk of the predictive model being biased or being overfitted to that specific type of chemical compound. This can, in some instances, negatively affect downstream training (e.g., it might be difficult to efficiently retrain the predictive model to accurately analyze test objects from different types of chemical compounds).

To illustrate how the feature vectors of test objects are used in clustering, consider the case in which a common set of ten features (the same ten features) within each feature vector are used for the clustering. In some embodiments, each test object in the test object dataset can have values for each of the ten features. In some embodiments, each test object of the test object dataset has measurement values for some of the features and the missing values are either filled in using imputation techniques or ignored (marginalized). In some embodiments, each test object of the test object dataset has values for some of the features and the missing values are filled in using constraints. The values from the feature vector of a test object in the test object dataset define the vector: X₁, X₂, X₃, X₄, X₅, X₆, X₇, X₈, X₉, X₁₀ where X, is the value of the i^{th} feature in the feature vector of a particular test object. If there are Q test objects in the test object dataset, selection of the 10 features can define Q vectors. In clustering, those members of the test object dataset that exhibit similar measurement patterns across their respective feature vectors tend to cluster together.

Particular exemplary clustering techniques that can be used include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, Jarvis-Patrick clustering, density-based spatial clustering algorithm, a divisive clustering algorithm, a supervised clustering algorithm, or ensembles thereof. Such clustering can be on the features within the feature vector of the respective test objects or the principal components (or other forms of reduction components) derived from them. In some embodiments, the clustering comprises unsupervised clustering where no preconceived notion of what clusters can form when the test object dataset is clustered are imposed.

Data clustering is an unsupervised process that requires optimization to be effective; for example, using either too few or too many clusters to describe a dataset can result in loss of information. See e.g., Jain et al. 1999 “Data Clustering: A review” AMC Computing Surveys 31(3), 264-323; and Berkhin 2002 “Survey of clustering datamining techniques” Tech Report, Accrue Software, San Jose, Calif., which are each hereby incorporated by reference. In some embodiments, to improve the clustering process, the plurality of test objects is normalized prior to clustering (e.g., one or more dimensions in each feature vector in the plurality of feature vectors is normalized (e.g., to a respective average value for the corresponding dimension as determined from the plurality of feature vectors).

In some embodiments, a centroid-based clustering algorithm is used to perform clustering of the plurality of test objects. Centroid-based clustering organizes the data into non-hierarchical clusters, and represents all of the objects in terms of central vectors (where the vectors themselves might not be part of the dataset). The algorithm then calculates the distance measure between each object and the central vectors and clusters the objects based on proximity to one of the central vectors. In some embodiments, Euclidian, Manhattan, or Minkowski distance measurements are used to calculate the distance measures between each test object and the central vectors. In some embodiments, a k-means, k-medoid, CLARA, or CLARANS clustering algorithm is used for clustering the plurality of test objects. Examples of k-means algorithms are described in Uppada 2014 “Centroid Based Clustering Algorithms—A Clarion Study” Int J Comp Sci and Inform Technol 5(6), 7309-7313, which is hereby incorporated by reference.

In some embodiments, a density-based clustering algorithm is used to perform clustering of the plurality of test objects. Density-based spatial clustering algorithms identify clusters as regions in a dataset (e.g., the plurality of feature vectors) of higher concentration (e.g., regions with high density of test objects). In some embodiments, density-based spatial clustering can be performed as described in Ester et al. 1996 “A Density-Based Algorithm for Discovering Clusters in Large Spatial Databases with Noise” KDD'96: Proceedings of the Second International Conference on Knowledge Discovery and Data Mining, 226-231, which is hereby incorporated by reference. In such embodiments, the algorithm allows for arbitrarily shaped distributions and does not assign outliers (e.g., test objects outside of concentrations of other test objects) to clusters.

In some embodiments, a hierarchical clustering (e.g., connectivity-based clustering) algorithm is used to perform clustering of the plurality of test objects. In general, hierarchical clustering is used to build a series of clusters and can be agglomerative or divisive as further described below (e.g., there are agglomerative or divisive subsets of hierarchical clustering methods). Rokach et al. for example, which is hereby incorporated by reference, describe various versions of agglomerative clustering methods (“Clustering Methods” 2005 Data Mining and Knowledge Discovery Handbook, 321-352).

In some embodiments, the hierarchical clustering comprises divisive clustering. Divisive clustering initially groups the plurality of test objects in one cluster and subsequently divides the plurality of test objects into more and more clusters (e.g., it is a recursive process) until a certain threshold (e.g., a number of clusters) is reached. Examples of different methods of divisive clustering are described for example in Chavent et al. 2007 “DIVCLUS-T: a monothetic divisive hierarchical clustering method” Comp Stats Data Anal 52 (2), 687-701; Sharma et al. 2017 “Divisive hierarchical maximum likelihood clustering” BMC Bioinform 18(Suppl 16):546; and Xiong et al. 2011 “DHCC: Divisive hierarchical clustering of categorical data” Data Min Knowl Disc doi 10.1007/s10618-011-0221-2, which are each hereby incorporated by reference.

In some embodiments, the hierarchical clustering comprises agglomerative clustering. Agglomerative clustering generally includes initially separating the plurality of test objects into multiple separate clusters (e.g., in some cases starting with individual test objects defining clusters) and merge pairs of clusters over successive iterations. Ward's method is an example of agglomerative clustering that uses the sum of squares to reduce variance between members of each cluster (e.g., it is a minimum variance agglomerative clustering technique). See Murtagh and Legendre 2014 “Ward's Hierarchical Agglomerative Clustering Method” J. Class 31, 274-295, which is hereby incorporated by reference. A drawback of many agglomerative clustering methods is their high computational requirements. In some embodiments, an agglomerative clustering algorithm can be combined with a k-means clustering algorithm. Non-limited examples of agglomerative and k-means clustering are described in Karthikeyan et al. 2020 “A comparative study of k-means clustering and agglomerative hierarchical clustering” Int J Emer Trends Eng Res 8(5), 1600-1604, which is hereby incorporated by reference. As an example, k-means clustering algorithms partition the plurality of test objects into discrete sets of k clusters (e.g., an initial k number of partitions) in the data space. In some embodiments, k-means clustering is applied to the plurality of test objects iteratively (e.g., k-means clustering is applied multiple times—for example consecutively—to the plurality of test objects). In some embodiments, the combined use of agglomerative and k-means clustering is less computationally demanding than either agglomerative or k-means clustering alone.

Block 216. Referring to block 216, in some embodiments, the target model is a convolutional neural network.

In some embodiments (e.g., when the at least one target object is a polymer with an active site and the test object is a chemical composition), a description of the test object posed against the respective target object is obtained by docking an atomic representation of the test object into an atomic representation of the active site of the polymer. Non-limiting examples of such docking are disclosed in Liu and Wang, 1999, “MCDOCK: A Monte Carlo simulation approach to the molecular docking problem,” Journal of Computer-Aided Molecular Design 13, 435-451; Shoichet et al., 1992, “Molecular docking using shape descriptors,” Journal of Computational Chemistry 13(3), 380-397; Knegtel et al., 1997 “Molecular docking to ensembles of protein structures,” Journal of Molecular Biology 266, 424-440, Morris et al., 2009, “AutoDock4 and AutoDockTools4: Automated Docking with Selective Receptor Flexibility,” J Comput Chem 30(16), 2785-2791; Sotriffer et al., 2000, “Automated docking of ligands to antibodies: methods and applications,” Methods: A Companion to Methods in Enzymology 20, 280-291; Morris et al., 1998, “Automated Docking Using a Lamarckian Genetic Algorithm and Empirical Binding Free Energy Function,” Journal of Computational Chemistry 19: 1639-1662; and Rarey et al., 1996, “A Fast Flexible Docking Method Using an Incremental Construction Algorithm,” Journal of Molecular Biology 261, 470-489, each of which is hereby incorporated by reference. Then a description of this pose of this respective test object to at least one target object is applied to the target model. In some such embodiments, the test object is a chemical compound, the respective target object comprises a polymer with a binding pocket, and the posing the description of the test object against the respective target object comprises docking modeled atomic coordinates for the chemical compound into atomic coordinates for the binding pocket.

In some embodiments, each test object is a chemical compound that is posed against one or more target objects and presented to the target model using any of the techniques disclosed in U.S. Pat. Nos. 10,546,237; 10,482,355; 10,002,312, and 9,373,059, each of which is hereby incorporated by reference.

In some embodiments, the convolutional neural network comprises an input layer, a plurality of individually weighted convolutional layers, and an output scorer, as described in U.S. Pat. No. 10,002,312, entitled “Systems and Methods for Applying a Convolutional Network to Spatial Data,” issued Jun. 19, 2018, which is hereby incorporated in its entirety. For example, in some such embodiments, the convolutional layers of the target model include an initial layer and a final layer. In some embodiments, the final layer may include gating using a threshold or activation function, f, which may be a linear or non-linear function. The activation function may be, for example, a rectified linear unit (ReLU) activation function, a Leaky ReLu activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

Responsive to input, in some embodiments, the input layer feeds values into the initial convolutional layer. Each respective convolutional layer, other than the final convolutional layer, in some embodiments, feeds intermediate values as a function of the weights of the respective convolutional layer and input values of the respective convolutional layer into another of the convolutional layers. The final convolutional layer, in some embodiments, feeds values into the scorer as a function of the final layer weights and input values. In this way, the scorer may score each of the feature vectors (e.g., an input vector as described in U.S. Pat. No. 10,002,312) describing a respective test object and these scores are collectively used to provide a corresponding target result (e.g., the classification described in U.S. Pat. No. 10,002,312) for each respective test object. In some embodiments, the scorer provides a respective single score for each of the feature vectors and the weighted average of these scores is used to provide a corresponding target result for each respective test object.

In some embodiments, the total number of layers used in a convolutional neural network (including input and output layers) ranges from about 3 to about 200. In some embodiments, the total number of layers is at least 3, at least 4, at least 5, at least 10, at least 15, or at least 20. In some embodiments, the total number of layers is at most 20, at most 15, at most 10, at most 5, at most 4, or at most 3. Those of skill in the art will recognize that the total number of layers used in the convolutional neural network may have any value within this range, for example, 8 layers.

In some embodiments, the total number of learnable or trainable parameters, e.g., weighting factors, biases, or threshold values, used in the convolutional neural network ranges from about 1 to about 10,000. In some embodiments, the total number of learnable parameters is at least 1, at least 10, at least 100, at least 500, at least 1,000, at least 2,000, at least 3,000, at least 4,000, at least 5,000, at least 6,000, at least 7,000, at least 8,000, at least 9,000, or at least 10,000. Alternatively, the total number of learnable parameters is any number less than 100, any number between 100 and 10,000, or a number greater than 10,000. In some embodiments, the total number of learnable parameters is at most 10,000, at most 9,000, at most 8,000, at most 7,000, at most 6,000, at most 5,000, at most 4,000, at most 3,000, at most 2,000, at most 1,000, at most 500, at most 100 at most 10, or at most 1. Those of skill in the art will recognize that the total number of learnable parameters used may have any value within this range.

Because convolutional neural networks require a fixed input size, some embodiments of the disclosed systems and methods that make use of a convolutional neural network for the target model crop the geometric data (the target object-test object complex) to fit within an appropriate bounding box. For example, a cube of 25-40 Å to a side, may be used. In some embodiments in which the target and/or test objects have been docketed into the active site of target objects, the center of the active site serves as the center of the cube.

While in some embodiments a square cube of fixed dimensions centered on the active site of the target object is used to partition the space into the voxel grid, the disclosed systems are not so limited. In some embodiments, any of a variety of shapes is used to partition the space into the voxel grid. In some embodiments, polyhedra, such as rectangular prisms, polyhedra shapes, etc. are used to partition the space.

In an embodiment, the grid structure may be configured to be similar to an arrangement of voxels. For example, each sub-structure may be associated with a channel for each atom being analyzed. Also, an encoding method may be provided for representing each atom numerically.

In some embodiments, the voxel map describing the interface between a test object and a target object takes into account the factor of time and may thus be in four dimensions (X, Y, Z, and time).

In some embodiments, other implementations such as pixels, points, polygonal shapes, polyhedrals, or any other type of shape in multiple dimensions (e.g. shapes in 3D, 4D, and so on) may be used instead of voxels.

In some embodiments, the geometric data is normalized by choosing the origin of the X, Y and Z coordinates to be the center of mass of a binding site of the target object as determined by a cavity flooding algorithm. For representative details of such algorithms, see Ho and Marshall, 1990, “Cavity search: An algorithm for the isolation and display of cavity-like binding regions,” Journal of Computer-Aided Molecular Design 4, pp. 337-354; and Hendlich et al., 1997, “Ligsite: automatic and efficient detection of potential small molecule-binding sites in proteins,” J. Mol. Graph. Model 15, no. 6, each of which is hereby incorporated by reference. Alternatively, in some embodiments, the origin of the voxel map is centered at the center of mass of the entire co-complex (of the test object bound to the target object, of just the target object, or of just the test object). The basis vectors may optionally be chosen to be the principal moments of inertia of the entire co-complex, of just the target object, or of just the test object. In some embodiments, the target object is a polymer having an active site, and the sampling samples the test object in each of the respective poses in the above-described plurality of different poses for the test object and the active site on the three-dimensional grid basis in which a center of mass of the active site is taken as the origin and the corresponding three dimensional uniform honeycomb for the sampling represents a portion of the polymer and the test object centered on the center of mass. In some embodiments, the uniform honeycomb is a regular cubic honeycomb and the portion of the polymer and the test object is a cube of predetermined fixed dimensions. Use of a cube of predetermined fixed dimensions, in such embodiments, ensures that a relevant portion of the geometric data is used and that each voxel map is the same size. In some embodiments, the predetermined fixed dimensions of the cube are N Å×NÅ×N Å, where N is an integer or real value between 5 and 100, an integer between 8 and 50, or an integer between 15 and 40. In some embodiments, the uniform honeycomb is a rectangular prism honeycomb and the portion of the polymer and the test object is a rectangular prism predetermined fixed dimensions Q Å×R Å×S Å, where Q is a first integer between 5 and 100, R is a second integer between 5 and 100, S is a third integer or real value between 5 and 100, and at least one number in the set {Q, R, S} is not equal to another value in the set {Q, R, S}.

In some embodiments, every voxel has one or more input channels, which may have various values associated with them, which in one implementation can be on/off, and may be configured to encode for a type of atom. Atom types may denote the element of the atom, or atom types may be further refined to distinguish between other atom characteristics. Atoms present may then be encoded in each voxel. Various types of encoding may be utilized using various techniques and/or methodologies. As an example encoding method, the atomic number of the atom may be utilized, yielding one value per voxel ranging from one for hydrogen to 118 for ununoctium (or any other element).

However, as discussed above, other encoding methods may be utilized, such as “one-hot encoding,” where every voxel has many parallel input channels, each of which is either on or off and encodes for a type of atom. Atom types may denote the element of the atom, or atom types may be further refined to distinguish between other atom characteristics. For example, SYBYL atom types distinguish single-bonded carbons from double-bonded, triple-bonded, or aromatic carbons. For SYBYL atom types, see Clark et al., 1989, “Validation of the General Purpose Tripos Force Field, 1989, J. Comput. Chem. 10, pp. 982-1012, which is hereby incorporated by reference.

In some embodiments, each voxel further includes one or more channels to distinguish between atoms that are part of the target object or cofactors versus part of the test object. For example, in one embodiment, each voxel further includes a first channel for the target object and a second channel for the test object. When an atom in the portion of space represented by the voxel is from the target object, the first channel is set to a value, such as “1”, and is zero otherwise (e.g., because the portion of space represented by the voxel includes no atoms or one or more atoms from the test object). Further, when an atom in the portion of space represented by the voxel is from the test object, the second channel is set to a value, such as “1”, and is zero otherwise (e.g., because the portion of space represented by the voxel includes no atoms or one or more atoms from the target object). Likewise, other channels may additionally (or alternatively) specify further information such as partial charge, polarizability, electronegativity, solvent accessible space, and electron density. For example, in some embodiments, an electron density map for the target object overlays the set of three-dimensional coordinates, and the creation of the voxel map further samples the electron density map. Examples of suitable electron density maps include, but are not limited to, multiple isomorphous replacement maps, single isomorphous replacement with anomalous signal maps, single wavelength anomalous dispersion maps, multi-wavelength anomalous dispersion maps, and 2F_{observable}−F_{calculated} maps. See McRee, 1993, Practical Protein Crystallography, Academic Press, which is hereby incorporated by reference.

In some embodiments, voxel encoding in accordance with the disclosed systems and methods may include additional optional encoding refinements. The following two are provided as examples.

In a first encoding refinement, the required memory may be reduced by reducing the set of atoms represented by a voxel (e.g., by reducing the number of channels represented by a voxel) on the basis that most elements rarely occur in biological systems. Atoms may be mapped to share the same channel in a voxel, either by combining rare atoms (which may therefore rarely impact the performance of the system) or by combining atoms with similar properties (which therefore could minimize the inaccuracy from the combination).

Another encoding refinement is to have voxels represent atom positions by partially activating neighboring voxels. This results in partial activation of neighboring neurons in the subsequent neural network and moves away from one-hot encoding to a “several-warm” encoding. For example, it may be illustrative to consider a chlorine atom, which has a van der Waals diameter of 3.5 Å and therefore a volume of 22.4 Å³ when a 1 Å³ grid is placed, voxels inside the chlorine atom will be completely filled and voxels on the edge of the atom will only be partially filled. Thus, the channel representing chlorine in the partially-filled voxels will be turned on proportionate to the amount such voxels fall inside the chlorine atom. For instance, if fifty percent of the voxel volume falls within the chlorine atom, the channel in the voxel representing chlorine will be activated fifty percent. This may result in a “smoothed” and more accurate representation relative to the discrete one-hot encoding. Thus, in some embodiments, the test object is a first compound and the target object is a second compound, a characteristic of an atom incurred in the sampling is spread across a subset of voxels in the respective voxel map and this subset of voxels comprises two or more voxels, three or more voxels, five or more voxels, ten or more voxels, or twenty-five or more voxels. In some embodiments, the characteristic of the atom consists of an enumeration of the atom type (e.g., one of the SYBYL atom types).

Thus, voxelation (rasterization) of the geometric data (the docking of a test object onto a target object) that has been encoded is based upon various rules applied to the input data.

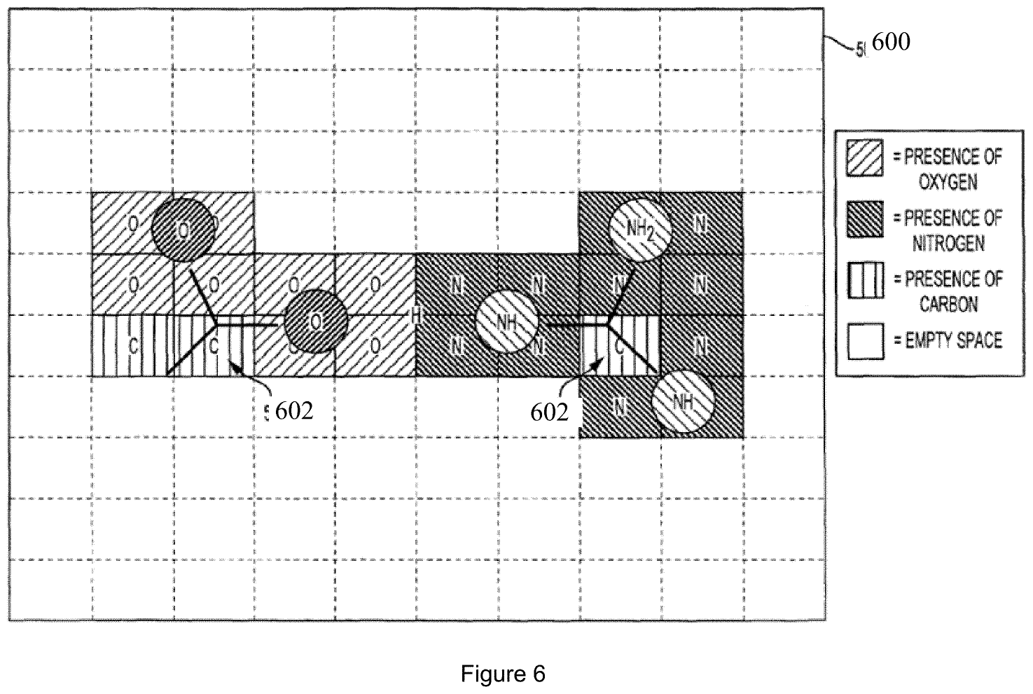

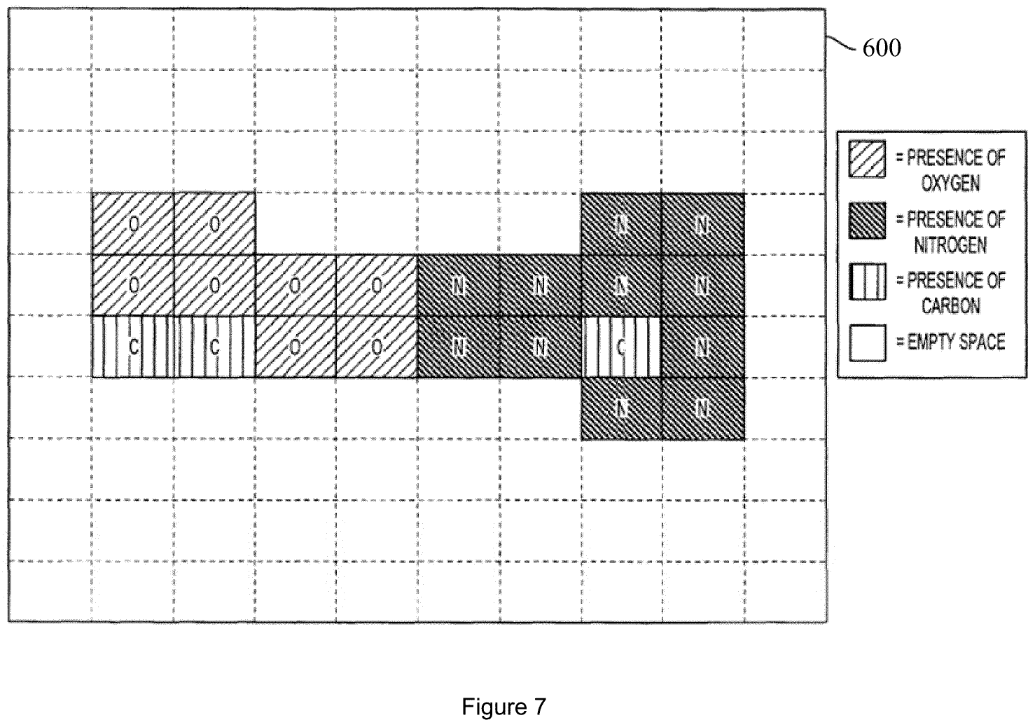

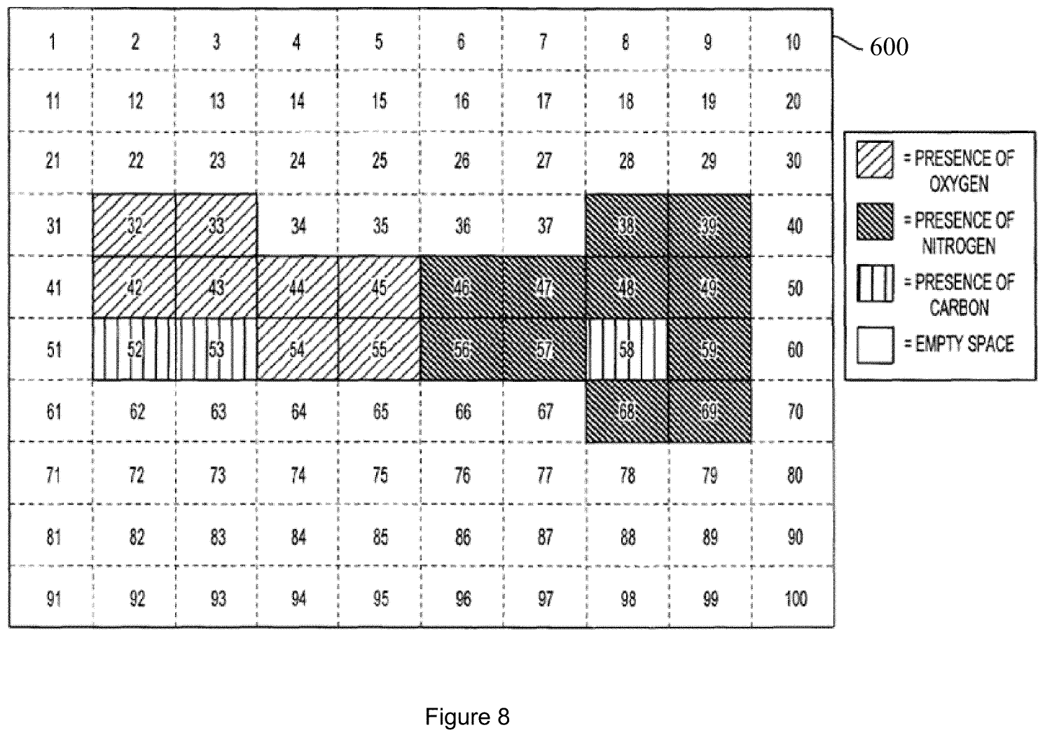

FIGS. 6 and 7 provide views of two test objects 602 encoded onto a two dimensional grid 600 of voxels, according to some embodiments. FIG. 6 provides the two test objects superimposed on the two dimensional grid. FIG. 7 provides the one-hot encoding, using the different shading patterns to respectively encode the presence of oxygen, nitrogen, carbon, and empty space. As noted above, such encoding may be referred to as “one-hot” encoding. FIG. 7 shows the grid 500 of FIG. 6 with the test objects 502 omitted. FIG. 8 provides a view of the two dimensional grid of voxels of FIG. 7, where the voxels have been numbered.

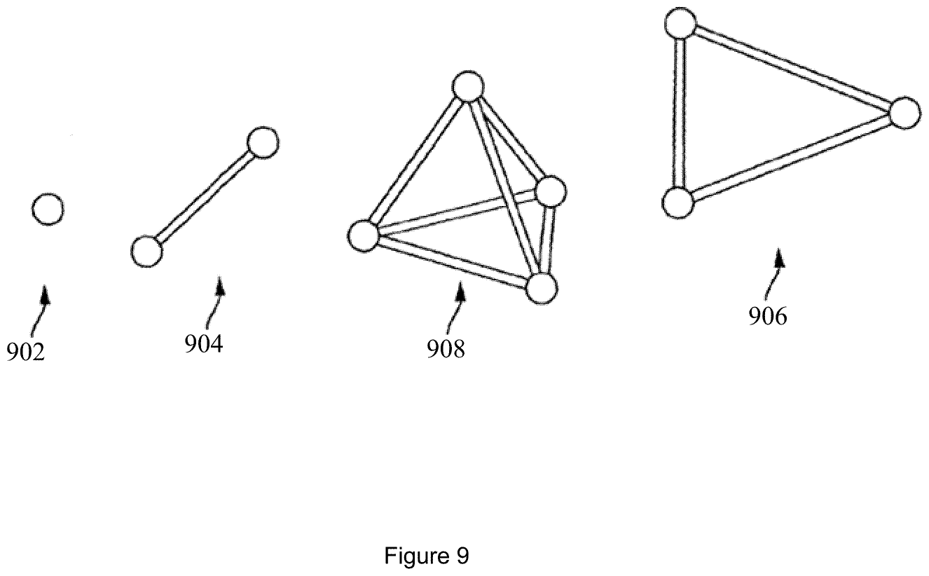

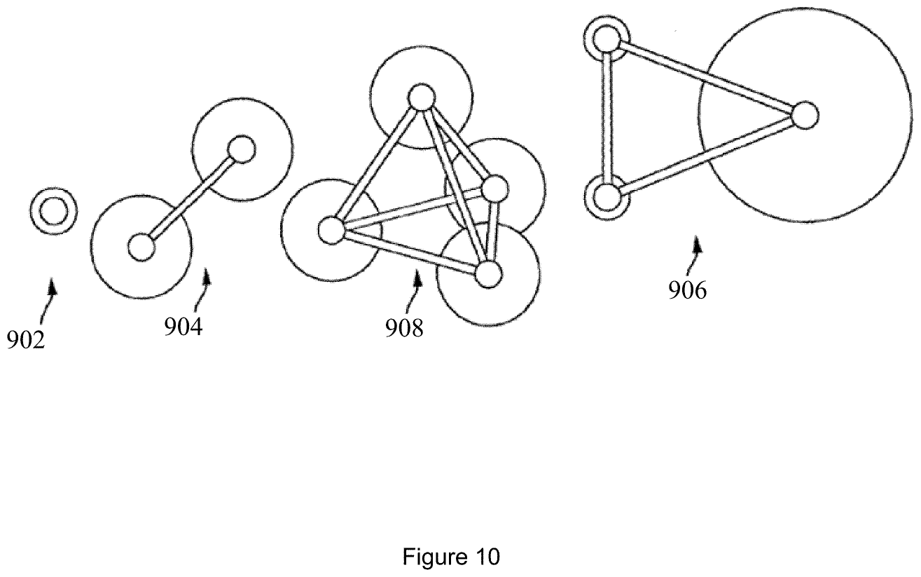

In some embodiments, feature geometry is represented in forms other than voxels. FIG. 9 provides view of various representations in which features (e.g., atom centers) are represented as 0-D points (representation 902), 1-D points (representation 904), 2-D points (representation 906), or 3-D points (representation 908). Initially, the spacing between the points may be randomly chosen. However, upon training the target model, the points may be moved closer together, or father apart. FIG. 10 illustrates a range of possible positions for each point.

In embodiments in which the interaction between a test object and target object is encoded as a voxel map, each voxel map is optionally unfolded into a corresponding vector, thereby creating a plurality of vectors, where each vector in the plurality of vectors is the same size. In some embodiments, each vector in the plurality of vectors is a one-dimensional vector. For instance, in some embodiments, a cube of 20 Å on each side is centered on the active site of the target object and is sampled with a three-dimensional fixed grid spacing of 1 Å to form corresponding voxels of a voxel map that hold in respective channels basic of the voxel structural features such as atom types as well as, optionally, more complex test object-target object descriptors, as discussed above. In some embodiments, the voxels of this three-dimensional voxel map are unfolded into a one-dimensional floating point vector. In some embodiments in which the target model is a convolutional neural network, the vectorized representation of voxel maps are subjected to a convolutional network.

In some embodiments, a convolutional layer in the plurality of convolutional layers comprises a set of filters (also termed kernels). Each filter has fixed three-dimensional size that is convolved (stepped at a predetermined step rate) across the depth, height and width of the input volume of the convolutional layer, computing a dot product (or other functions) between entries (weights) of the filter and the input thereby creating a multi-dimensional activation map of that filter. In some embodiments, the filter step rate is one element, two elements, three elements, four elements, five elements, six elements, seven elements, eight elements, nine elements, ten elements, or more than ten elements of the input space. Thus, consider the case in which a filter has size 5³. In some embodiments, this filter will compute the dot product (or other mathematical function) between a contiguous cube of input space that has a depth of five elements, a width of five elements, and a height of five elements, for a total number of values of input space of 125 per voxel channel.

The input space to the initial convolutional layer (e.g., the output from the input layer) is formed from either a voxel map or a vectorized representation of the voxel map. In some embodiments, the vectorized representation of the voxel map is a one-dimensional vectorized representation of the voxel map that serves as the input space to the initial convolutional layer. Nevertheless, when a filter convolves its input space and the input space is a one-dimensional vectorized representation of the voxel map, the filter still obtains from the one-dimensional vectorized representation those elements that represent a corresponding contiguous cube of fixed space in the target object−test object complex. In some embodiments, the filter uses standard bookeeping techniques to select those elements from within the one-dimensional vectorized representation that form the corresponding contiguous cube of fixed space in the target object−test object complex. Thus, in some instances, this necessarily involves taking a non-contiguous subset of element in the one-dimensional vectorized representation in order to obtain the element values of the corresponding contiguous cube of fixed space in the target object−test object complex.

In some embodiments, the filter is initialized (e.g., to Gaussian noise) or trained to have 125 corresponding weights (per input channel) in which to take the dot product (or some other form of mathematical operation such as the function of the 125 input space values in order to compute a first single value (or set of values) of the activation layer corresponding to the filter. In some embodiment the values computed by the filter are summed, weighted, and/or biased. To compute additional values of the activation layer corresponding to the filter, the filter is then stepped (convolved) in one of the three dimensions of the input volume by the step rate (stride) associated with the filter, at which point the dot product or some other form of mathematical operation between the filter weights and the 125 input space values (per channel) is taken at the new location in the input volume is taken. This stepping (convolving) is repeated until the filter has sampled the entire input space in accordance with the step rate. In some embodiments, the border of the input space is zero padded to control the spatial volume of the output space produced by the convolutional layer. In typical embodiments, each of the filters of the convolutional layer canvas the entire three-dimensional input volume in this manner thereby forming a corresponding activation map. The collection of activation maps from the filters of the convolutional layer collectively form the three-dimensional output volume of one convolutional layer, and thereby serves as the three-dimensional (three spatial dimensions) input of a subsequent convolutional layer. Every entry in the output volume can thus also be interpreted as an output of a single neuron (or a set of neurons) that looks at a small region in the input space to the convolutional layer and shares parameters with neurons in the same activation map. Accordingly, in some embodiments, a convolutional layer in the plurality of convolutional layers has a plurality of filters and each filter in the plurality of filters convolves (in three spatial dimensions) a cubic input space of N³ with stride Y, where N is an integer of two or greater (e.g., 2, 3, 4, 5, 6, 7, 8, 9, 10, or greater than 10) and Y is a positive integer (e.g. 1, 2, 3, 4, 5, 6, 7, 8, 9, 10, or greater than 10).

Each layer in the plurality of convolutional layers is associated with a different set of weights. With more particularity, each layer in the plurality of convolutional layers includes a plurality of filters and each filter comprises an independent plurality of weights. In some embodiments, a convolutional layer has 128 filters of dimension 5³ and thus the convolutional layer has 128×5×5×5 or 16,000 weights per channel in the voxel map. Thus, if there are five channels in the voxel map, the convolutional layer will have 16,000×5 weights, or 80,000 weights. In some embodiments some or all such weights (and, optionally, biases) of every filter in a given convolutional layer may be tied together, e.g. constrained to be identical.

Responsive to input of a respective vector in the plurality of vectors, the input layer feeds a first plurality of values into the initial convolutional layer as a first function of values in the respective vector.

Each respective convolutional layer, other than the final convolutional layer, feeds intermediate values, as a respective second function of (i) the different set of weights associated with the respective convolutional layer and (ii) input values received by the respective convolutional layer, into another convolutional layer in the plurality of convolutional layers. For instance, each respective filter of the respective convolutional layer canvasses the input volume (in three spatial dimensions) to the convolutional layer in accordance with the characteristic three-dimensional stride of the convolutional layer and at each respective filter position, takes the dot product (or some other mathematical function) of the filter weights of the respective filter and the values of the input volume (contiguous cube that is a subset of the total input space) at the respect filter position thereby producing a calculated point (or a set of points) on the activation layer corresponding to the respective filter position. The activation layers of the filters of the respective convolutional layer collectively represent the intermediate values of the respective convolutional layer.

The final convolutional layer feeds final values, as a third function of (i) the different set of weights associated with the final convolutional layer and (ii) input values received by the final convolutional layer, into the scorer. For instance, each respective filter of the final convolutional layer canvasses the input volume (in three spatial dimensions) to the final convolutional layer in accordance with the characteristic three-dimensional stride of the convolutional layer and at each respective filter position, takes the dot product (or some other mathematical function) of the filter weights of the filter and the values of the input volume at the respect filter position thereby calculating a point (or a set of points) on the activation layer corresponding to the respective filter position. The activation layers of the filters of the final convolutional layer collectively represent the final values that are fed to scorer.

In some embodiments, the convolutional neural network has one or more activation layers. In some embodiments, the activation layer is a layer of neurons that applies the non-saturating activation function f(x)=max(0, x). It increases the nonlinear properties of the decision function and of the overall network without affecting the receptive fields of the convolution layer. In other embodiments, the activation layer has other functions to increase nonlinearity, for example, the saturating hyperbolic tangent function f(x)=tanh, f(x)=|tanh(x)|, and the sigmoid function f(x)=(1+e^{-x})⁻¹. Nonlimiting examples of other activation functions found in other activation layers in some embodiments for the neural network may include, but are not limited to, logistic (or sigmoid), softmax, Gaussian, Boltzmann-weighted averaging, absolute value, linear, rectified linear, bounded rectified linear, soft rectified linear, parameterized rectified linear, average, max, min, some vector norm LP (for p=1, 2, 3, . . . , ∞), sign, square, square root, multiquadric, inverse quadratic, inverse multiquadric, polyharmonic spline, and thin plate spline.

In some embodiments, zero or more of the layers a target model (in embodiments in which the target model is a convolutional neural network) may consist of pooling layers. As in a convolutional layer, a pooling layer is a set of function computations that apply the same function over different spatially-local patches of input. For pooling layers, the output is given by a pooling operators, e.g. some vector norm LP for p=1, 2, 3, . . . , ∞, over several voxels. Pooling is typically done per channel, rather than across channels. Pooling partitions the input space into a set of three-dimensional boxes and, for each such sub-region, outputs the maximum. The pooling operation provides a form of translation invariance. The function of the pooling layer is to progressively reduce the spatial size of the representation to reduce the amount of parameters and computation in the network, and hence to also control overfitting. In some embodiments a pooling layer is inserted between successive convolutional layers in a target model that is in the form of a convolutional neural network. Such a pooling layer operates independently on every depth slice of the input and resizes it spatially. In addition to max pooling, the pooling units can also perform other functions, such as average pooling or even L2-norm pooling.

In some embodiments, zero or more of the layers in a target model (in embodiments in which the target model is a convolutional neural network) may consist of normalization layers, such as local response normalization or local contrast normalization, which may be applied across channels at the same position or for a particular channel across several positions. These normalization layers may encourage variety in the response of several function computations to the same input.

In some embodiments, the scorer (in embodiments in which the target model is a convolutional neural network) comprises a plurality of fully-connected layers and an evaluation layer where a fully-connected layer in the plurality of fully-connected layers feeds into the evaluation layer. Neurons in a fully connected layer have full connections to all activations in the previous layer, as seen in regular neural networks. Their activations can hence be computed with a matrix multiplication followed by a bias offset. In some embodiments, each fully connected layer has 512 hidden units, 1024 hidden units, or 2048 hidden units. In some embodiments there are no fully connected layers, one fully connected layer, two fully connected layers, three fully connected layers, four fully connected layers, five fully connected layers, six or more fully connected layers or ten or more fully connected layers in the scorer.

In some embodiments, the evaluation layer discriminates between a plurality of activity classes. In some embodiments, the evaluation layer comprises a logistic regression cost layer over a two activity classes, three activity classes, four activity classes, five activity classes, or six or more activity classes.

In some embodiments, the evaluation layer comprises a logistic regression cost layer over a plurality of activity classes. In some embodiments, the evaluation layer comprises a logistic regression cost layer over a two activity classes, three activity classes, four activity classes, five activity classes, or six or more activity classes.

In some embodiments, the evaluation layer discriminates between two activity classes and the first activity classes (first classification) represents an IC₅₀, EC₅₀, Kd, or KI for the test object with respect to the target object that is above a first binding value, and the second activity class (second classification) is an IC₅₀, EC₅₀, Kd, or KI for the test object with respect to the target object that is below the first binding value. In some such embodiments the target result is an indication that the test object has the first activity or the second activity. In some embodiments, the first binding value is one nanomolar, ten nanomolar, one hundred nanomolar, one micromolar, ten micromolar, one hundred micromolar, or one millimolar.

In some embodiments, the evaluation layer comprises a logistic regression cost layer over two activity classes and the first activity classes (first classification) represents an IC₅₀, EC₅₀, Kd, or KI for the test object with respect to the target object that is above a first binding value, and the second activity class (second classification) is an IC₅₀, EC₅₀, Kd, or KI for the test object with respect to the target object that is below the first binding value. In some such embodiments the target result is an indication that the test object has the first activity or the second activity. In some embodiments, the first binding value is one nanomolar, ten nanomolar, one hundred nanomolar, one micromolar, ten micromolar, one hundred micromolar, or millimolar.

In some embodiments, the evaluation layer discriminates between three activity classes and the first activity classes (first classification) represents an IC₅₀, EC₅₀, Kd, or KI for the test object with respect to the target object that is above a first binding value, the second activity class (second classification) is an IC₅₀, EC₅₀, Kd, or KI for the test object with respect to the target object that is between the first binding value and a second binding value, and the third activity class (third classification) is an IC₅₀, EC₅₀, Kd, or KI for the test object with respect to the target object that is below the second binding value, where the first binding value is other than the second binding value. In some such embodiments the target result is an indication that the test object has the first activity, the second activity, or the third activity.

In some embodiments, the evaluation layer comprises a logistic regression cost layer over three activity classes and the first activity classes (first classification) represents an IC₅₀, EC₅₀, Kd, or KI for the test object with respect to the target object that is above a first binding value, the second activity class (second classification) is an IC₅₀, EC₅₀, Kd, or KI for the test object with respect to the target object that is between the first binding value and a second binding value, and the third activity class (third classification) is an IC₅₀, EC₅₀, Kd, or KI for the test object with respect to the target object that is below the second binding value, where the first binding value is other than the second binding value. In some such embodiments the target result is an indication that the test object has the first activity, the second activity, or the third activity.

In some embodiments, the scorer (in embodiments in which the target model is a convolutional neural network) comprises a fully connected single layer or multilayer perceptron. In some embodiments the scorer comprises a support vector machine, random forest, nearest neighbor. In some embodiments, the scorer assigns a numeric score indicating the strength (or confidence or probability) of classifying the input into the various output categories. In some cases, the categories are binders and nonbinders or, alternatively, the potency level (IC₅₀, EC₅₀ or KI potencies of e.g., <1 molar, <1 millimolar, <100 micromolar, <10 micromolar, <1 micromolar, <100 nanomolar, <10 nanomolar, <1 nanomolar). In some such embodiments the target result is an indication is an identification of one of these categories for the test object.

Details for obtaining a target result from a target model for a complex between a test object and a target object have been described above. As discussed above, in some embodiments, each test object is docked into a plurality of poses with respect to the target object. To present all such poses at once to the target model may require a prohibitively large input field (e.g., an input field of size equal to number of voxels*number of channels*number of poses in the case where the target model is a convolutional neural network). While in some embodiments all poses are concurrently presented to the target model, in other embodiments each such pose is processed into a voxel map, vectorized, and serves as sequential input into the target model (e.g., when the target model is a convolutional neural network). In this way, a plurality of scores are obtained from the target model, where each score in the plurality of scores corresponds to the input of a vector in the plurality of vectors into the input layer of the scorer of the target model. In some embodiments, the scores for each of the poses of a given test object with a given target object are combined together (e.g., as a weighted mean of the scores, as a measure of central tendency of the scores, etc.) to produce a final target result for a respective test object.

In some embodiments where the scorer output of a target model is numeric, the outputs may be combined using any of the activation functions described herein or that are known or developed. Examples include, but are not limited to, a non-saturating activation function f(x)=max(0,x), a saturating hyperbolic tangent function f(x)=tanh, f(x)=|tanh(x)|, the sigmoid function f(x)=(1+e^{-x})⁻¹, logistic (or sigmoid), softmax, Gaussian, Boltzmann-weighted averaging, absolute value, linear, rectified linear, bounded rectified linear, soft rectified linear, parameterized rectified linear, average, max, min, some vector norm LP (for p=1, 2, 3, . . . , ∞), sign, square, square root, multiquadric, inverse quadratic, inverse multiquadric, polyharmonic spline, and thin plate spline.

In some embodiments of the present disclosure, the target model may be configured to utilize the Boltzmann distribution to combine outputs, as this matches the physical probability of poses if the outputs are interpreted as indicative of binding energies. In other embodiments of the present disclosure, the max( ) function may also provide a reasonable approximation to the Boltzmann and is computationally efficient.

In some embodiments where the scorer output of the target model is not numeric, the scorer may be configured to combine the outputs using various ensemble voting schemes, which may include, as illustrative, non-limiting examples, majority, weighted averaging, Condorcet methods, Borda count, among others, to form the corresponding target result.

In some embodiments, the system may be configured to apply an ensemble of scorers, e.g., to generate indicators of binding affinity.

In some embodiments, the test object is a chemical compound and using the plurality of scores (from the plurality of poses for the test object) to characterize (e.g. determine a classification) of the test object comprises taking a measure of central tendency of the plurality of scores. When the measure of central tendency satisfies a predetermined threshold value or predetermined threshold value range, the test object is deemed to have a first classification. When the measure of central tendency fails to satisfy the predetermined threshold value or predetermined threshold value range, the test object is deemed to have a second classification. In some such embodiments, the target result outputted by the target model for the respective test object is an indication of one of these classifications.

In some embodiments, the using the plurality of scores to characterize the test object comprises taking a weighted average of the plurality of scores (from the plurality of poses for the test object). When the weighted average satisfies a predetermined threshold value or predetermined threshold value range, the test object is deemed to have a first classification. When the weighted average fails to satisfy the predetermined threshold value or predetermined threshold value range, the test object is deemed to have a second classification. In some embodiments, the weighted average is a Boltzman average of the plurality of scores. In some embodiments, the first classification is an IC₅₀, EC₅₀, Kd, or KI for the test object with respect to the target object that is above a first binding value (e.g., one nanomolar, ten nanomolar, one hundred nanomolar, one micromolar, ten micromolar, one hundred micromolar, or one millimolar) and the second classification is an IC₅₀, EC₅₀, Kd, or KI for the test object with respect to the target object that is below the first binding value. In some such embodiments, the target result outputted by the target model for the respective test object is an indication of one of these classifications.

In some embodiments, the using the plurality of scores to provide a target result for the test object comprises taking a weighted average of the plurality of scores (from the plurality of poses for the test object). When the weighted average satisfies a respective threshold value range in a plurality of threshold value ranges, the test object is deemed to have a respective classification in a plurality of a respective classifications that uniquely corresponds to the respective threshold value range. In some embodiments, each respective classification in the plurality of classifications is an IC₅₀, EC₅₀, Kd, or KI range (e.g., between one micromolar and ten micromolar, between one nanomolar and 100 nanomolar) for the test object with respect to the target object.

In some embodiments, a single pose for each respective test object against a given target object is run through the target model and the respective score assigned by the target model for each of the respective test objects on this basis is used to classify the test objects.

In some embodiments, the weighted mean average of the target model scores of one or more poses of a test object against each of a plurality of target objects evaluated by the target model using the techniques disclosed herein is used to provide a target result for the test object. For instance, in some embodiments, the plurality of target objects are taken from a molecular dynamics run in which each target object in the plurality of target objects represents the same polymer at a different time step during the molecular dynamics run. A voxel map of each of one or more poses of the test object against each of these target objects is evaluated by the target model to obtain a score for each independent pose−target object pair and the weighted mean average of these scores, or some other measure of central tendency of these scores is used to provide a target result for the target object.

Block 218. Referring to block 218 of FIG. 2A, in some embodiments, the at least one target object is a single object (e.g., each target object is a respective single object). In some embodiments, the single object is a polymer. In some embodiments, the polymer comprises an active site (e.g., the polymer is an enzyme with an active site). In some embodiments, the polymer is a protein, a polypeptide, a polynucleic acid, a polyribonucleic acid, a polysaccharide, or an assembly of any combination thereof. In some embodiments, the single object is an organometallic complex. In some embodiments, the single object is a surfactant, a reverse micelle, or liposome.

In some embodiments, each test object in the plurality of test object comprises a respective chemical compound that may or may not bind to an active site of at least one target object with corresponding affinity (e.g., an affinity for forming chemical bonds to the at least one target object).

In some embodiments, the at least one target object comprises at least two target objects, at least three target objects, at least four target objects, at least five target objects, or at least six target objects. In some embodiments, each target object is a respective single object (e.g., a single protein, a single polypeptide, etc.), as described above. In some embodiments, one or more target objects of the at least one target object comprises multiple objects (e.g., a protein complex and/or an enzyme with multiple subunits such as a ribosome).

Block 220. Referring to block 220 of FIG. 2B, the method proceeds by training a predictive model in an initial state using at least i) the subset of test objects as independent variables and ii) the corresponding subset of target results as dependent variables, thereby updating the predictive model to an updated trained state. That is, the predictive model is trained to predict what the target result (target model score) would be for a given test compound without incurring the computational expense of the target model. Moreover, in some embodiments, the predictive model does not make use of the at least one target object. In such embodiments, the predictive model attempts to predict the score of the target model simply based on the information provided for the test object in the test object dataset (e.g., the chemical structure of the test object) and not the interaction between the test object and the one or more target objects.

Referring to block 222, in some embodiments, the target model exhibits a first computational complexity in evaluating respective test objects, the predictive model exhibits a second computational complexity in evaluating respective test objects, and the second computational complexity is less than the first computational complexity (e.g., the predictive model requires less time and/or less computational effort to provide a respective predictive result for a test object than the target model requires to provide a corresponding target result for the same test object).

As used herein, the phrase “computational complexity” is interchangeable with the phrase “time complexity” and is related to a required amount of time needed to obtain a result upon application of a model to a test object and at least one target object with a given number of processors and is also related to a required number of processors needed to obtain a result upon application of a model to a test object and at least one target object within a given amount of time, where each processor has a given amount of processing power. As such, computational complexity as used herein refers to prediction complexity of a model. However, in some embodiments, the target model exhibits a first training computational complexity, the predictive model exhibits a second training computational complexity, and the second training computational complexity is less than the first training computational complexity as well. Table 2 below lists some exemplary predictive models and their estimated computational complexity for making predictions (prediction complexity):

TABLE 2

| Predictive Model | Prediction Complexity |
|---|---|
| Decision Tree | O(p) |
| Random Forest | O(pnₜᵣₑₑₛ) |
| Linear Regression | O(p) |
| Support Vector Machine (Kernel) | O(nₛᵥp) |
| k-Nearest Neighbors | O(np) |
| Naïve Bayes | O(p) |

In Table 2,p is the number of features of the test object evaluated by the classifier in providing a classifier result, nₜᵣₑₑₛ is the number of trees (for methods based on various trees), and O refers to the Bachmann-Landau notation that refers to the upper bound of the growth rate of the function. See, for example, Arora and Barak, 2009, Computational Complexity: A Modern Approach, Cambridge University Press, Cambridge England. By contrast, one estimate of the total time complexity of a convolutional neural network, which is one form of a training model, is:

$$O ( {\sum }_{l = 1}^{d} {n}_{l - 1} \cdot {s}_{l}^{2} \cdot {n}_{l} \cdot {m}_{l}^{2} )$$

where l is the index of a convolutional layer, d is the depth (number of convolutional layers), nₗ is the number of filters (also known as “width”) in the l^{th} layer (nₗ₋₁ is also known as the number of input channels of the l^{th} layer), sₗ is the spatial size (length) of the filter, mₗ is the spatial size of the output feature map. This time complexity applies to both training and testing time, though with a different scale. The training time per test object is roughly three times of the testing time per test object (one for forward propagation and two for backward propagation). See, Hi and Sun, 2014, “Convolutional Neural Networks at Constrained Time Cost,” arXiv:1412.1710v1 [cs.CV] 4 Dec. 2014, which is hereby incorporated by reference. Thus, clearly, the time complexity of the convolutional neural network is greater than that of the time complexity of the example predictive models provided in Table 1.

Block 224. Referring to block 224 of FIG. 2B, in some embodiments the predictive model in the initial trained state comprises an untrained or partially trained classifier. For instance, in some embodiments the predictive model is partially trained on test objects, or other forms of data, such as assay data not represented in the test object dataset, separate and apart from the data provided from the plurality of test objects in the test object dataset using, for example, transfer learning techniques. In one example, the predictive model is partially trained on the binding affinity data of a set of compounds, where such compounds may or may not be in the test object dataset using transfer learning techniques.

Referring to block 226, in some embodiments, the predictive model in the updated trained state comprises an untrained or partially trained classifier that is distinct from the predictive model in the initial trained state (e.g., one or more weights of the predictive model have been altered). The ability to retrain, or update, an existing classifier is particularly useful when the training dataset is subject to change (e.g., in cases where the training dataset increases in size and/or in number of classes).

In some embodiments, a boosting algorithm is used to update (train) the predictive model. Boosting algorithms are generally described by Dai et al. 2007 “Boosting for transfer learning” in Proc 24^{th} Int Conf on Mach Learn, which is hereby incorporated by reference. Boosting algorithms can include reweighting data (e.g., a subset of the test objects) that has been previously used to train a predictive model when new data (e.g., an additional subset of the test objects) is added to the dataset used to retrain or update a predictive model. See e.g., Freund et al. 1997 “A decision-theoretic generalization of on-line learning and an application to boosting” J Computer and System Sciences 55(1), 119-139, which is hereby incorporated by reference.

In some embodiments, as discussed above, depending on the type of algorithm (e.g., for when the predictive model is not a single decision tree) that is used for the predictive model in the initial trained state, a transfer learning method is used to update the predictive model to an updated trained state (e.g., upon each successive iteration of the method). Transfer learning generally involves the transfer of knowledge from a first model to a second model (e.g., knowledge either from a first set of tasks or from a first dataset to a second set of tasks or a second dataset). Additional reviews of transfer learning methods can be found in Torrey et al. 2009 “Transfer Learning” in the Handbook of Research on Machine Learning Applications; Pan et al. 2009 “A Survey on Transfer Learning” IEEE Transactions on Knowledge and Data Engineering doi:10.1109/TKDE.2009.191; and Molochanov et al. 2016 “Pruning Convolutional Neural Networks for Resource Efficient Transfer Learning” arXiv:1611.06440v1 which are each hereby incorporated by reference. In some embodiments, a variant of a random forest can be used with a dynamic training dataset. See Ristin et al. 2014 IEEE Conference on Computer Vision and Pattern Recognition (CVPR), 3654-3661, which is hereby incorporated by reference.

In some embodiments, the predictive model comprises a random forest tree, a random forest comprising a plurality of multiple additive decision trees, a neural network, a graph neural network, a dense neural network, a principal component analysis, a nearest neighbor analysis, a linear discriminant analysis, a quadratic discriminant analysis, a support vector machine, an evolutionary method, a projection pursuit, regression, a Naïve Bayes algorithm, or ensembles thereof.

Random forest, decision tree, and boosted tree algorithms. Decision trees are described generally by Duda, 2001, Pattern Classification, John Wiley & Sons, Inc., New York, 395-396, which is hereby incorporated by reference. A random forest is generally defined as a collection of decision trees. Tree-based methods partition the feature space into a set of rectangles, and then fit a model (such as a constant) in each rectangle. In some embodiments, the decision tree comprises random forest regression. One specific algorithm that can be used for the predictive model is classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, 396-408 and 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests in general are described in Breiman, 1999, Technical Report 567, Statistics Department, U.C. Berkeley, September 1999, which is hereby incorporated by reference in its entirety.

Neural networks, graph neural networks, dense neural networks. Various neural networks may be employed as either or both the target model and/or the predictive model provided that the predictive model has less computational complexity than the target model. Neural network algorithms, including convolutional neural network (CNN) algorithms, are disclosed in e.g., Vincent et al., 2010, J Mach Learn Res 11, 3371-3408; Larochelle et al., 2009, J Mach Learn Res 10, 1-40; and Hassoun, 1995, Fundamentals of Artificial Neural Networks, Massachusetts Institute of Technology, each of which is hereby incorporated by reference. In some embodiments, another variation of a neural network algorithm—including but not exclusive to graph neural networks (GNNs) and dense neural networks (DNNs)—is used for the predictive model. Graph neural networks are useful for data that is represented in non-Euclidean space (e.g., particularly datasets with high complexity). Overviews of GNNs are provided by Wu et al. 2019 “A Comprehensive Survey on Graph Neural Networks” arVix:1901.00596; and Zhou et al 2018 “Graph Neural Networks: A Review of Methods and Applications” arVix:1812.08434. GNNs can be combined with other data analysis methods to enable drug discovery. See e.g., Altre-Tran et al. 2017 “Low Data Drug Discovery with One-Shot Learning” ACS Cent Sci 3, 283-293. Dense neural networks generally include a high number of neurons in each layer and are described in Montavon et al. 2018 “Methods for interpreting and understanding deep neural networks” Digit Signal Process 73, 1-15; and Finnegan et al. 2017 “Maximum entropy methods for extracting the learned features of deep neural networks” PLoS Comput Biol. 13(10), 1005836, each of which is hereby incorporated by reference.

Principal component analysis. Principal component analysis is one of several methods that are often used for dimensionality reduction of complex data (e.g., to reduce the number of objects under consideration). Examples of using PCA for data clustering are provided, for example, by Yeung and Ruzzo 2001 “Principal component analysis for clustering gene expression data” Bioinformat 17(9), 763-774, which is hereby incorporated by reference. Principal components are typically ordered by the extent of variance present (e.g., only the first n components are considered to convey signal instead of noise) and are uncorrelated (e.g., each component is orthogonal to other components).

Nearest neighbor analysis. Nearest neighbor analysis is typically performed with Euclidean distances. Examples of nearest neighbor analysis are provided by Weinberger et al. 2006 “Distance metric learning for large margin nearest neighbor classification” in NIPS MIT Press 2, 3. Nearest neighbor analysis is beneficial because in some embodiments it is effective in settings with large training datasets. See Sonawane 2015 “A Review on Nearest Neighbour Techniques for Large Data” International Journal of Advances Research in Computer and Communication Engineering 4(11), 459-461, which is hereby incorporated by reference.

Linear discriminant analysis. Linear discriminant analysis (LDA) is typically performed to identify a linear combination of features that characterize or separate classes of test objects. Examples of LDA are provided by Ye et al. 2004 “Two-Dimensional Linear Discriminant Analysis” Advances in Neural Information Processing Systems 17, 1569-1576, Prince et al. 2007 “Probabilistic Linear Discriminant Analysis for Inferences about Identity” 11th International Conference on Computer Vision, 1-8. LDA is beneficial because it can be applied both to large and small sample size, and it can be used in high dimensions. See Kaipatnen 1997 “Utilizing Geometric Anomalies of High Dimension: When Complexity Makes Computation Easier” Computer-Intensive Methods in Control and Signal Processing, 283-294.

Quadratic discriminant analysis. Quadratic discriminant analysis (QDA) is closely related to LDA, but in QDA an individual covariance matrix is estimated for every class of objects. See Wu et al. 1996 “Comparison of regularized discriminant analysis, linear discriminant analysis and quadratic discriminant analysis, applied to NIR data” Analytica Chimica Acta 329, 257-265. Examples of QDA are provided by Zhang 1997 “Identification of protein coding regions in the human genome by quadratic discriminant analysis” PNAS 94, 565-568; Zhang et al. 2003 “Splice site prediction with quadratic discriminant analysis using diversity measure” Nuc Acids Res 31(21), 6124-6220, each of which is hereby incorporated by reference. QDA is beneficial because it provides a greater number of effective parameters than LDA, as described in Wu et al. 1996 “Comparison of regularized discriminant analysis, linear discriminant analysis and quadratic discriminant analysis, applied to NIR data” Analytica Chimica Acta 329, 257-265, which is hereby incorporated by reference.

Support vector machines. Non-limiting examples of support vector machine (SVM) algorithms are described in Cristianini and Shawe-Taylor, 2000 “An Introduction to Support Vector Machines,” Cambridge University Press; Boser et al., 1992, “A training algorithm for optimal margin classifiers,” in Proceedings of the 5^{th} Annual ACM Workshop on Computational Learning Theory, ACM Press, Pittsburgh, Pa., 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary-labeled data training set with a hyper-plane that is maximally distant from the labeled data. For cases in which no linear separation is possible, SVMs can work in combination with the technique of ‘kernels,’ which automatically realizes a non-linear mapping to a feature space. The hyper-plane found by the SVM in feature space corresponds to a non-linear decision boundary in the input space.

Linear regression. As used herein, linear regression can encompass simple, multiple, and/or multivariate linear regression analysis. Linear regression uses linear approach to modeling the relationship between a dependent variable (also known as scalar response) and one or more independent variables (also known as explanatory variables) and as such can be used as a predictive model in the present disclosure. See Altman et al. 2015 “Simple Linear Regression” Nature Methods 12, 999-1000, which is hereby incorporated by reference. The relationships are predicted using linear predictor functions, whose parameters are estimated form the data using linear models. In some embodiments, simple linear regression is used to model the relationship between a dependent variable and a single independent variable. An example of simple linear regression can be found in Altman et al. 2015 “Simple Linear Regression” Nature Methods 12, 999-1000, which is hereby incorporated by reference.

In some embodiments, multiple linear regression is used to model the relationship between a dependent variable and multiple independent variables and as such can be used as a predictive model in the present disclosure. An example of multiple linear regression can be found in Sousa et al. 2007 “Multiple linear regression and artificial neural networks based on principal components to predict ozone concentration” Environ Model & Soft 22(1), 97-103, which is hereby incorporated by reference. In some embodiments, multivariate linear regression is used to model the relationship between multiple dependent variables and any number of independent variables. A non-limiting example of multivariate linear regression can be found in Wang et al. 2016 “Discriminative Feature Extraction via Multivariate Linear Regression for SSVEP-Based BCI” IEEE Transactions on Neural Systems and Rehabilitation Engineering 24(5), 532-541, which is hereby incorporated by reference.

Naïve Bayes algorithms. Naive Bayes classifiers (algorithms) are a family of “probabilistic classifiers” based on applying Bayes' theorem with strong (naïve) independence assumptions between the features. In some embodiments, they are coupled with Kernel density estimation. See, Hastie, Trevor, 2001, The elements of statistical teaming: data mining, inference, and prediction, Tibshirani, Robert, Friedman, J. H. (Jerome H.), New York: Springer, which is hereby incorporated by reference.

In some embodiments, the training of the predictive model in an initial state using at least i) the subset of test objects as independent variables of the predictive model and ii) the corresponding subset of target results as dependent variables of the predictive model further comprises using iii) the at least one target object as an independent variable in order to update the predictive model to an updated trained state.

Blocks 228-230. Referring to block 228 of FIG. 2B, the method proceeds by applying the predictive model in an updated trained state (e.g., a retrained predictive model) to the full plurality of test objects, thereby obtaining an instance of a plurality of predictive results. Referring to block 230, in some embodiments, the instance of the plurality of predictive results includes a respective predictive result for each test object in the plurality of test objects. In this way, a balance is achieved between the high computational burden of the target model, and its commensurate improved performance, and the lower computational burden of the predictive model, and its commensurate inferior performance. The target model is used to obtain target results for just a subset of the test objects thereby forming a training set for training the predictive model. This training set is presumably more accurate due to the performance of the more computationally burdensome target model as well as the fact that it makes use of an interaction between at least one target object and the test objects. For instance, in some embodiments, a target object is an enzyme with an active site and the target model scores the interaction between each test object in the subset of test objects and the target object. The training set is then used to train the predictive model. As such, in typical embodiments, the predictive model is trained using the training set, which comprises target model scores for each test object in the subset of test objects and the chemical data provides for each such test object in the test object dataset, so that the predictive model can predict the score of the target model without using the target object (e.g., without docking the test objects to the target object). Then the predictive model, now trained, is applied against the full plurality of test objects to obtain an instance of a plurality of predictive results. The instance of predictive results comprises the score the trained predictive model predicts would be the target model score for each object in the full plurality of target objects. In this way, the performance of the more computationally burdensome target model, with its concomitant docking, is fully leveraged to assist in reducing the number of test objects in the test dataset. Moreover, the efficiency of the predictive model is fully leveraged to obtain a test result for each of the test objects in order to reduce the number of test objects in the test dataset.

Blocks 232-234. Referring to block 232 of FIG. 2B, the method proceeds by eliminating a portion of the test objects from the plurality of test objects based at least in part on the instance of the plurality of predictive results (e.g., in accordance with any of the elimination criteria described below). In some embodiments, the applying the target model, for each respective test object in a subset of test objects from the plurality of test objects, to the respective test object and at least one target object to obtain the corresponding target result, thereby obtaining a corresponding subset of target results (block 210), the training the predictive model in an initial trained state (block 220), the applying the predictive model in the updated trained state to the plurality of test objects thereby obtaining an instance of a plurality of predictive results (block 228), and the eliminating a portion of the test objects from the plurality of test objects based at least in part on the instance of the plurality of predictive results (block 232) is an iterative process that is repeated a number of times (e.g., 2 times, 3 times, more than 3 times, more than ten times, more than fifteen times, etc.), subject to the evaluation performed described in block 236 below. Each time the process is repeated (in each iteration), a portion of the test objects remaining in the plurality of test objects is removed from the plurality of test objects based at least in part on the latest instance of the plurality of predictive results from block 228.

Referring to block 234, in some embodiments, the eliminating comprises i) clustering the plurality of test objects, thereby assigning each test object in the plurality of test objects to a respective cluster in a plurality of clusters, and ii) eliminating a subset of test objects from the plurality of test objects based at least in part on a redundancy of test objects in individual clusters in the plurality of clusters (e.g., to ensure a variety of different chemical compounds in the plurality of test objects). In other words, in such embodiments, in each iteration of block 232, the remaining plurality of test objects are clustered. In some embodiments, this clustering is based on the feature vectors of the test objects as described above. In some embodiments, any of the clustering described in block 214 may be used to perform the clustering of block 234. Whereas in block 214 such clustering was performed to select a subset of test objects for use against the target model, in block 234 the clustering is performed to permanently eliminate test objects from the plurality of test objects. Consider an example in which the clustering of block 234 clusters the test objects remaining in the plurality test objects into Q clusters, where Q is a positive integer of 2 or greater (e.g., 2, 3, 4, 5, 6, 7, 8, 9, 10, more than 10, more than 20, more than 30, more than 100, etc.). In some such embodiments, the same number of test objects in each of these clusters is kept in the plurality of test objects and all other test objects are removed from the plurality of test objects. In this way, the test objects remaining in the plurality of test objects is balanced across all the clusters.

The plurality of predictive results produced in step 232 represent the scores that the predictive model predicts the target model would call for the plurality of test objects.

If the scoring is done in a scheme in which lower scores represent compounds that have better affinity for the one or more target objects, than it is of interest to remove those test objects that have higher scores. Thus, in some alternative embodiments clustering is not used and the eliminating of block 232 comprises i) ranking the plurality of test objects based on the instance of the plurality of predictive results, and ii) removing from the plurality of test objects those test objects in the plurality of test objects that fail to have a corresponding prediction score that satisfies a threshold cutoff (e.g., so as to ensure that test objects remaining in the plurality of test objects have high prediction scores). In some embodiments, the threshold cutoff is a top threshold percentage (e.g., a percentage of the plurality of test objects that are most highly ranked based on the plurality of predictive results). In some such embodiments, the top threshold percentage represents the test objects in the plurality of test objects whose predictive results are in the top 90 percent, the top 80 percent, the top 75 percent, the top 60 percent, the top 50 percent, the top 40 percent, the top 30 percent, the top 25 percent, the top 20 percent, the top 10 percent, or the top 5 percent of the plurality of predictive results. In such embodiments, the corresponding bottom percentage of test objects are eliminated from the plurality of test objects for further consideration (e.g., thereby reducing the number of test objects in the plurality of test objects).

If the scoring is done in a scheme in which higher scores represent compounds that have better affinity for the one or more target objects, than it is of interest to remove those test objects that have lower scores. Thus, in some alternative embodiments clustering is not used and the eliminating of block 232 comprises i) ranking the plurality of test objects based on the instance of the plurality of predictive results, and ii) removing from the plurality of test objects those test objects in the plurality of test objects that fail to have a corresponding prediction score that satisfies a threshold cutoff (e.g., so as to ensure that test objects remaining in the plurality of test objects have low prediction scores). In some such embodiments, the threshold cutoff is a bottom threshold percentage (e.g., a percentage of the plurality of test objects that are least highly ranked based on the plurality of predictive results). In some embodiments, the bottom threshold percentage represents the test objects in the plurality of test objects whose predictive results are in the bottom 90 percent, the bottom 80 percent, the bottom 75 percent, the bottom 60 percent, the bottom 50 percent, the bottom 40 percent, the bottom 30 percent, the bottom 25 percent, the bottom 20 percent, the bottom 10 percent, or the bottom 5 percent of the plurality of predictive results. In such embodiments, the corresponding top percentage of test objects are eliminated from the plurality of test objects for further consideration (e.g., thereby reducing the number of test objects in the plurality of test objects).

In some embodiments, each instance of the eliminating (e.g., in embodiments where the method repeats eliminating a portion of the test objects from the plurality of test objects) eliminates between one tenth and nine tenths of the test objects in the plurality of test objects at the particular iteration of block 232. In some embodiments, each instance of the eliminating eliminates more than five percent, more than ten percent, more than fifteen percent, more than twenty percent or more than twenty-five percent of the test objects present in the plurality of test objects at the particular iteration of block 232.

In some embodiments, each instance of the eliminating eliminates between five percent and thirty percent, between ten percent and forty percent, between fifteen percent and seventy percent, between twenty percent and fifty percent, between twenty-five percent and ninety percent of the plurality of test objects at the particular iteration of block 232. In some embodiments, each instance of the eliminating eliminates between one quarter and three quarters of the test objects in the plurality of test objects at the particular iteration of block 232. In some embodiments, each instance of the eliminating eliminates between one quarter and one half of the test objects in the plurality of test objects at the particular iteration of block 232.

In some embodiments, each instance of the eliminating (block 232) eliminates a predetermined number (or portion) of test objects from the plurality of test objects. For example, in some embodiments, each respective instance of the eliminating (block 232) eliminates five percent of the test objects that are in the plurality of test objects at the respective instance of the eliminating. In some embodiments, one or more instances of the eliminating eliminates a different number (or portion) of test objects. For example, initial instances of the eliminating (block 232) may eliminate a higher percentage of the plurality of test objects that are in the plurality of test objects during these initial instances of the eliminating 232 while subsequent instances of the eliminating may eliminate a lower percentage of the plurality of test objects that are in the plurality of test objects during these subsequent instances of the eliminating 232. For instance, eliminating 10 percent of the plurality of test compounds in initial instances while eliminating 5 percent of the plurality of test compounds in subsequent instances. In another example, initial instances of the eliminating (block 232) may eliminate a lower percentage of the plurality of test objects that are in the plurality of test objects during these initial instances of the eliminating while subsequent instances of the eliminating may eliminate a higher percentage of the plurality of test objects that are in the plurality of test objects during these subsequent instances of the eliminating 232. For instance, eliminating 5 percent of the plurality of test compounds in initial instances of the eliminating while eliminating 10 percent of the plurality of test compounds in subsequent instances of the eliminating 232.

Block 236. Referring to block 236 of FIG. 2C, the method proceeds by determining whether one or more predefined reduction criteria are satisfied. When the one or more predefined reduction criteria are not satisfied the method further comprises the following. The target model is applied (i) for each respective test object in an additional subset of test objects in the plurality of test objects, to the respective test object and at least one target object to obtain a corresponding target result, thereby obtaining an additional subset of target results. The additional subset of test objects is selected at least in part on the instance of the plurality of predictive results. The subset of test objects is updated (ii) by incorporating the additional subset of test objects into the subset of test objects (e.g., the previous subset of test objects). The subset of target results is updated (iii) by incorporating the additional subset of target results into the subset of target results. Thus, the subset of target results grows as the method progressive iterates between running the target model, training the predictive model, and running the predictive model. The predictive model is modified (iv), after the updating (ii) and the updating (iii), by applying the predictive model to at least 1) the subset of test objects as independent variables and 2) the corresponding subset of target results as corresponding dependent variables, thereby providing the predictive model in an updated trained state. The applying (block 228), eliminating (block 232), and determining (block 236) are repeated until one or more predefined reduction criteria are satisfied.

In some embodiments, modifying (iv) the predictive model comprises either retraining or training a new partially trained predictive model.

In some embodiments, when the one or more predefined reduction criteria are satisfied, the method further comprises i) clustering the plurality of test objects, thereby assigning each test object in the plurality of test objects to a cluster in a plurality of clusters, and ii) eliminating one or more test objects from the plurality of test objects based at least in part on redundancy of test objects in individual clusters in the plurality of clusters.

In some embodiments, clustering the plurality of test objects is performed as described with regard to block 212.

Referring to block 238, in some embodiments, the applying (i) further comprises forming the additional subset of test objects by selecting one or more test objects from the plurality of test objects based on evaluation of one or more features selected from the plurality of feature vectors, as described above (e.g., by selecting test objects from a variety of clusters).

In some embodiments, the additional subset of test objects is of a same or similar size as the subset of test objects. In some embodiments, the additional subset of test objects is of a different size as the subset of test objects. In some embodiments, the additional subset of test objects is distinct from the subset of test objects.

In some embodiments, the additional subset of test objects comprises at least 1,000 test objects, at least 5,000 test objects, at least 10,000 test objects, at least 25,000 test objects, at least 50,000 test objects, at least 75,000 test objects, at least 100,000 test objects, at least 250,000 test objects, at least 500,000 test objects, at least 750,000 test objects, at least 1 million test objects, at least 2 million test objects, at least 3 million test objects, at least 4 million test objects, at least 5 million test objects, at least 6 million test objects, at least 7 million test objects, at least 8 million test objects, at least 9 million test objects, or at least 10 million test objects.

In some embodiments, the modifying (iv) the predictive model comprises retraining the predictive model (e.g., rerunning the training process on an updated subset of test objects and potentially changing some parameters or hyperparameters of the predictive model). In some embodiments, the modifying (iv) the predictive model comprises training a new predictive model (e.g., to replace the previous predictive model).

In some embodiments, the modifying (iv) further comprises using 3) the at least one target object as an independent variable, in addition to using at least 1) the subset of test objects as independent variables and 2) the corresponding subset of target results as corresponding dependent variables. In other words, in some embodiments the predictive model does, in fact, dock the test objects to the target object in order to generate predictive results that are trained against the target results of the target model, provided that the predictive model, with docking, remains computationally less burdensome than the target model with its concomitant binding.

Referring to block 240, in some embodiments, satisfaction of the one or more predefined reduction criteria comprises correlating the plurality of predictive results to the corresponding target results from the subset of target results. For instance in some embodiments the one or more predefined reduction criteria are satisfied when the correlation between the plurality of predictive results and the corresponding target results is 0.60 or greater, 0.65 or greater, 0.70 or greater, 0.75 or greater, 0.80 or greater, 0.85 or greater or 0.90 or greater.

Referring to block 240, in some embodiments, satisfaction of the one or more predefined reduction criteria comprises determining an average difference between the plurality of predictive results and the corresponding target results on an absolute or normalized scale and, with the one or more predefined reduction criteria being satisfied when this average difference less than a threshold amount. In such embodiments the threshold amount is application dependent.

In some embodiments, satisfaction of the one or more predefined reduction criteria comprises determining that the number of test objects in the plurality of test objects has dropped below a threshold number of objects. In some embodiments, the one or more predefined reduction criteria require the plurality of test objects to have no more than 30 test objects, no more than 40 test objects, no more than 50 test objects, no more than 60 test objects, no more than 70 test objects, no more than 90 test objects, no more than 100 test objects, no more than 200 test objects, no more than 300 test objects, no more than 400 test objects, no more than 500 test objects, no more than 600 test objects, no more than 700 test objects, no more than 800 test objects, no more than 900 test objects, or no more than 1000 test objects.

In some embodiments, the one or more predefined reduction criteria require the plurality of test objects to have between 2 and 30 test objects, between 4 and 40 test objects, between 5 and 50 test objects, between 6 and 60 test objects, between 5 and 70 test objects, between 10 and 90 test objects, between 5 and 100 test objects, between 20 and 200 test objects, between 30 and 300 test objects, between 40 and 400 test objects, between 40 and 500 test objects, between 40 and 600 test objects, or between 50 and 700 test objects.

In some embodiments, satisfaction of the one or more predefined reduction criteria comprises determining that the number of test objects in the plurality of test objects has been reduced by a threshold percentage of the number of test objects in the test object database. In some embodiments, the one or more predefined reduction criteria require that the plurality of test objects be reduced by at least 10% of the test object database, at least 20% of the test object database, at least 30% of the test object database, at least 40% of the test object database, at least 50% of the test object database, at least 60% of the test object database, at least 70% of the test object database, at least 80% of the test object database, at least 90% of the test object database, at least 95% of the test object database, or at least 99% of the test object database.

In some embodiments, the one or more predefined reduction criteria is a single reduction criterion. In some embodiments, the one or more predefined reduction criteria is a single reduction criterion and this single reduction criterion is any one of the reduction criterion described in the present disclosure.

In some embodiments, the one or more predefined reduction criteria is a combination of reduction criteria. In some embodiments, this combination of reduction criteria is any combination of the reduction criteria described in the present disclosure.

Referring to block 242, in some embodiments, when the one or more predefined reduction criterion are satisfied, the method further comprises applying the predictive model to the plurality of test objects and the at least one target object, thereby causing the predictive model to provide a respective score for each test object in the plurality of test objects (e.g., each score is for a respective test object and the target object). In some such embodiments, each respective score corresponds to an interaction between a respective test object and the at least one target object. In some embodiments, each score is used to characterize the at least one target object. In some embodiments, the score refers to a binding affinity (e.g., between a respective test object with one or more target objects) as described in U.S. Pat. No. 10,002,312, entitled “Systems and Methods for Applying a Convolutional Network to Spatial Data,” which is hereby incorporated in its entirety. In some embodiments, interaction between a test object and a target object is affected by the distance, angle, atom type, molecular charge and/or polarization, and surrounding stabilizing or destabilizing environmental factors.

In some alternative embodiments, when the one or more predefined reduction criterion are satisfied, the method further comprises applying the target model to the remaining plurality of test objects and the at least one target object, thereby causing the target model to provide a respective target score for each remaining test object in the plurality of test objects (e.g., each target score is for a respective test object and a target object in the one or more target objects). In some such embodiments, each respective target score corresponds to an interaction between a respective test object and the at least one target object. In some embodiments, each target score is used to characterize the at least one target object. In some embodiments, the target score refers to a binding affinity (e.g., between a respective test object with one or more target objects) as described in U.S. Pat. No. 10,002,312, entitled “Systems and Methods for Applying a Convolutional Network to Spatial Data,” which is hereby incorporated in its entirety. In some embodiments, interaction between a test object and a target object is affected by the distance, angle, atom type, molecular charge and/or polarization, and surrounding stabilizing or destabilizing environmental factors.

### Example 1—Use Cases

The following are sample use cases provided for illustrative purposes only that describe some applications of some embodiments of the invention. Other uses may be considered, and the examples provided below are non-limiting and may be subject to variations, omissions, or may contain additional elements.

While each example below illustrates binding affinity prediction, the examples may be found to differ in whether the predictions are made over a single molecule, a set, or a series of iteratively modified molecules; whether the predictions are made for a single target or many, whether activity against the targets is to be desired or avoided, and whether the important quantity is absolute or relative activity; or, if the molecules or targets sets are specifically chosen (e.g., for molecules, to be existing drugs or pesticides; for proteins, to have known toxicities or side-effects).

Hit discovery. Pharmaceutical companies spend millions of dollars on screening compounds to discover new prospective drug leads. Large compound collections are tested to find the small number of compounds that have any interaction with the disease target of interest. Unfortunately, wet lab screening suffers experimental errors and, in addition to the cost and time to perform the assay experiments, the gathering of large screening collections imposes significant challenges through storage constraints, shelf stability, or chemical cost. Even the largest pharmaceutical companies have only between hundreds of thousands to a few millions of compounds, versus the tens of millions of commercially available molecules and the hundreds of millions of simulate-able molecules.

A potentially more efficient alternative to physical experimentation is virtual high throughput screening. In the same manner that physics simulations can help an aerospace engineer to evaluate possible wing designs before a model is physically tested, computational screening of molecules can focus the experimental testing on a small subset of high-likelihood molecules. This may reduce screening cost and time, reduces false negatives, improves success rates, and/or covers a broader swath of chemical space.

In this application, a protein target may serve as the target object. A large set of molecules may also be provided in the form of the test object dataset. For each test object that remains upon application of the disclosed methods, a binding affinity is predicted against the protein target. The resulting scores may be used to rank the remaining molecules, with the best-scoring molecules being most likely to bind the target protein. Optionally, the ranked molecule list may be analyzed for clusters of similar molecules; a large cluster may be used as a stronger prediction of molecule binding, or molecules may be selected across clusters to ensure diversity in the confirmatory experiments.

Off-target side-effect prediction. Many drugs may be found to have side-effects. Often, these side-effects are due to interactions with biological pathways other than the one responsible for the drug's therapeutic effect. These off-target side-effects may be uncomfortable or hazardous and restrict the patient population in which the drug's use is safe. Off-target side effects are therefore an important criterion with which to evaluate which drug candidates to further develop. While it is important to characterize the interactions of a drug with many alternative biological targets, such tests can be expensive and time-consuming to develop and run. Computational prediction can make this process more efficient.

In applying an embodiment of the invention, a panel of biological targets may be constructed that are associated with significant biological responses and/or side-effects. The system may then be configured to predict binding against each protein in the panel in turn by treating each such protein as a target object. Strong activity (that is, activity as potent as compounds that are known to activate the off-target protein) against a particular target may implicate the molecule in side-effects due to off-target effects.

Toxicity prediction. Toxicity prediction is a particularly-important special case of off-target side-effect prediction. Approximately half of drug candidates in late stage clinical trials fail due to unacceptable toxicity. As part of the new drug approval process (and before a drug candidate can be tested in humans), the FDA requires toxicity testing data against a set of targets including the cytochrome P450 liver enzymes (inhibition of which can lead to toxicity from drug-drug interactions) or the hERG channel (binding of which can lead to QT prolongation leading to ventricular arrhythmias and other adverse cardiac effects).

In toxicity prediction, the system may be configured to constrain the off-target proteins to be key antitargets (e.g. CYP450, hERG, or 5-HT_{2B} receptor). The binding affinity for a drug candidate may then be predicted against these proteins by treating each of these proteins as a target object (e.g. in separate independent runs). Optionally, the molecule may be analyzed to predict a set of metabolites (subsequent molecules generated by the body during metabolism/degradation of the original molecule), which can also be analyzed for binding against the antitargets. Problematic molecules may be identified and modified to avoid the toxicity or development on the molecular series may be halted to avoid wasting additional resources.

Agrochemical design. In addition to pharmaceutical applications, the agrochemical industry uses binding prediction in the design of new pesticides. For example, one desideratum for pesticides is that they stop a single species of interest, without adversely impacting any other species. For ecological safety, a person could desire to kill a weevil without killing a bumblebee.

For this application, the user could input a set of protein structures as the one or more target objects, from the different species under consideration, into the system. A subset of proteins could be specified as the proteins against which to be active, while the rest would be specified as proteins against which the molecules should be inactive. As with previous use cases, some set of molecules (whether in existing databases or generated de novo) would be considered against each target object as test objects, and the system would return the molecules with maximal effectiveness against the first group of proteins while avoiding the second.

### CONCLUSION

Plural instances may be provided for components, operations, or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A method for reducing a number of test objects in a plurality of test objects in a test object dataset, the method comprising:
A) obtaining, in electronic format, the test object dataset;
B) applying a target model, for each respective test object in a subset of test objects from the plurality of test objects, to the respective test object and at least one target object to obtain a corresponding target result, thereby obtaining a corresponding subset of target results;
C) training a predictive model in an initial trained state using at least i) the subset of test objects as independent variables and ii) the corresponding subset of target results as dependent variables, thereby updating the predictive model to an updated trained state;
D) applying the predictive model in an updated trained state to the plurality of test objects thereby obtaining an instance of a plurality of predictive results;
E) eliminating a portion of the test objects from the plurality of test objects based at least in part on the instance of the plurality of predictive results; and
F) determining whether one or more predefined reduction criteria are satisfied, wherein, when the one or more predefined reduction criteria are not satisfied, the method further comprises:
(i) applying the target model, for each respective test object in an additional subset of test objects from the plurality of test objects, to the respective test object and the at least one target object to obtain a corresponding target result, thereby obtaining an additional subset of target results, wherein the additional subset of test objects is selected at least in part on the instance of the plurality of predictive results;
(ii) updating the subset of test objects by incorporating the additional subset of test objects into the subset of test objects;
(iii) updating the subset of target results by incorporating the additional subset of target results into the subset of target results;
(iv) modifying, after the updating (ii) and the updating (iii), the predictive model by applying the predictive model to at least 1) the subset of test objects as a plurality of independent variables of the predictive model and 2) the corresponding subset of target results as a corresponding plurality of dependent variables of the predictive model, thereby providing the predictive model in an updated trained state; and
(v) repeating the applying (D), eliminating (E), and determining (F), wherein the plurality of test objects, before application of an instance of the eliminating E), comprises at least 100 million test objects.

2. The method of claim 1, wherein
the target model exhibits a first computational complexity,
the predictive model exhibits a second computational complexity, and
the second computational complexity is less than the first computational complexity.

3. The method of either claim 1 or claim 2, wherein the test object dataset includes a plurality of feature vectors, wherein each feature vector is for a respective test object in the plurality of test objects.

4. The method of any one of claims 1-3, wherein the applying B) further comprises randomly selecting one or more test objects from the plurality of test objects to form the subset of test objects.

5. The method of claim 3, wherein the applying B) further comprises selecting one or more test objects from the plurality of test objects for the subset of test objects based on evaluation of one or more features selected from the plurality of feature vectors.

6. The method of claim 3, wherein each feature vector in the plurality of feature vectors is a one-dimensional vector.

7. The method of either claim 3 or claim 4, wherein the applying F)(i) further comprises forming the additional subset of test objects by selecting one or more test objects from the plurality of test objects based on evaluation of one or more features selected from the plurality of feature vectors.

8. The method of any one of claims 1-7, wherein satisfaction of the one or more predefined reduction criteria comprises comparing each predictive result in the plurality of predictive results to a corresponding target result from the subset of target results.

9. The method of any one of claims 1-7, wherein satisfaction of the one or more predefined reduction criteria comprises determining that the number of test objects in the plurality of test objects has dropped below a threshold number of objects.

10. The method of any one of claims 1-9, wherein the target model is a convolutional neural network.

11. The method of any one of claims 1-9, wherein the predictive model comprises a random forest tree, a random forest comprising a plurality of multiple additive decision trees, a neural network, a graph neural network, a dense neural network, a principal component analysis, a nearest neighbor analysis, a linear discriminant analysis, a quadratic discriminant analysis, a support vector machine, an evolutionary method, a projection pursuit, a linear regression, a Naïve Bayes algorithm, a multi-category logistic regression algorithm, or ensembles thereof.

12. The method of any one of claims 1-11, wherein
the at least one target object is a single object, and
the single object is a polymer.

13. The method of claim 12 wherein the polymer comprises an active site.

14. The method of claim 12 or 13, wherein the polymer is a protein, a polypeptide, a polynucleic acid, a polyribonucleic acid, a polysaccharide, or an assembly of any combination thereof.

15. The method of claim 12, wherein the polymer is applied to the target model based upon a set of three-dimensional coordinates {x₁, . . . , x_{N}} for a crystal structure of the polymer resolved at a resolution of 2.5 Å or better.

16. The method of claim 12, wherein the polymer is applied to the target model based upon a set of three-dimensional coordinates {x₁, . . . , x_{N}} for a crystal structure of the polymer resolved at a resolution of 3.3 Å or better.

17. The method of claim 12, wherein the polymer is applied to the target model based upon spatial coordinates that are an ensemble of three-dimensional coordinates for the polymer determined by nuclear magnetic resonance, neutron diffraction, or cryo-electron microscopy.

18. The method of any one of claims 1-19, wherein the plurality of test objects, before application of an instance of the eliminating E), comprises at least 500 million test objects, at least 1 billion test objects, at least 2 billion test objects, at least 3 billion test objects, at least 4 billion test objects, at least 5 billion test objects, at least 6 billion test objects, at least 7 billion test objects, at least 8 billion test objects, at least 9 billion test objects, at least 10 billion test objects, at least 11 billion test objects, at least 15 billion test objects, at least 20 billion test objects, at least 30 billion test objects, at least 40 billion test objects, at least 50 billion test objects, at least 60 billion test objects, at least 70 billion test objects, at least 80 billion test objects, at least 90 billion test objects, at least 100 billion test objects, or at least 110 billion test objects.

19. The method of claim 0, wherein the one or more predefined reduction criteria require the plurality of test objects to have no more than 30 test objects, no more than 40 test objects, no more than 50 test objects, no more than 60 test objects, no more than 70 test objects, no more than 90 test objects, no more than 100 test objects, no more than 200 test objects, no more than 300 test objects, no more than 400 test objects, no more than 500 test objects, no more than 600 test objects, no more than 700 test objects, no more than 800 test objects, no more than 900 test objects, or no more than 1000 test objects.

20. The method of any one of claims 1-19, wherein each test object in the plurality of test objects represents a chemical compound.

21. The method of any one of claims 1-20, wherein the predictive model in the initial trained state comprises an untrained or partially trained classifier.

22. The method of any one of claims 1-21, wherein the predictive model in the updated trained state comprises an untrained or a partially trained classifier that is distinct from the predictive model in the initial trained state.

23. The method of any one of claims 1-22, wherein the subset of test objects comprises at least 1,000 test objects, at least 5,000 test objects, at least 10,000 test objects, at least 25,000 test objects, at least 50,000 test objects, at least 75,000 test objects, at least 100,000 test objects, at least 250,000 test objects, at least 500,000 test objects, at least 750,000 test objects, at least 1 million test objects, at least 2 million test objects, at least 3 million test objects, at least 4 million test objects, at least 5 million test objects, at least 6 million test objects, at least 7 million test objects, at least 8 million test objects, at least 9 million test objects, or at least 10 million test objects.

24. The method of any one of claims 1-23, wherein the additional subset of test objects comprises at least 1,000 test objects, at least 5,000 test objects, at least 10,000 test objects, at least 25,000 test objects, at least 50,000 test objects, at least 75,000 test objects, at least 100,000 test objects, at least 250,000 test objects, at least 500,000 test objects, at least 750,000 test objects, at least 1 million test objects, at least 2 million test objects, at least 3 million test objects, at least 4 million test objects, at least 5 million test objects, at least 6 million test objects, at least 7 million test objects, at least 8 million test objects, at least 9 million test objects, or at least 10 million test objects.

25. The method of either claim 23 or 24, wherein the additional subset of test objects is distinct from the subset of test objects.

26. The method of claim 1, wherein the F) modifying (iv) the predictive model comprises retraining the predictive model.

27. The method of claim 1, wherein the training (C) further comprises using iii) the at least one target object as an independent variable of the predictive model, in addition to using the at least i) the subset of test objects as a plurality of independent variables of the predictive model, and ii) the corresponding subset of target results as a plurality of dependent variables of the predictive model.

28. The method of either claim 1 or claim 27, wherein the at least one target object comprises at least two target objects, at least three target objects, at least four target objects, at least five target objects, or at least six target objects.

29. The method of claim 1, wherein the instance of the plurality of predictive results includes a respective predictive result for each test object in the plurality of test objects.

30. The method of any one of claims 1-29, wherein the modifying F)(iv) further comprises using 3) the at least one target object as an independent variable, in addition to using at least 1) the subset of test objects as independent variables and 2) the corresponding subset of target results as corresponding dependent variables of the predictive model.

31. The method of any one of claims 1-30, wherein, when the one or more predefined reduction criteria are satisfied, the method further comprises:
i) clustering the plurality of test objects, thereby assigning each test object in the plurality of test objects to a cluster in a plurality of clusters; and
ii) eliminating one or more test objects from the plurality of test objects based at least in part on redundancy of test objects in individual clusters in the plurality of clusters.

32. The method of any one of claims 1-30, the method further comprising selecting the subset of test objects from the plurality of test objects by:
i) clustering the plurality of test objects thereby assigning each test object in the plurality of test objects to a respective cluster in a plurality of clusters, and
ii) selecting the subset of test objects from the plurality of test objects based at least in part on a redundancy of test objects in individual clusters in the plurality of clusters.

33. The method of any one of claims 1-32, wherein, when the one or more predefined reduction criterion are satisfied, the method further comprises applying the predictive model to the plurality of test objects and the at least one target object, thereby causing the predictive model to provide a respective interaction score for each test object in the plurality of test objects.

34. The method of claim 33, wherein each respective interaction score corresponds to an interaction between a respective test object and the at least one target object.

35. The method of either claim 33 or 34, wherein each respective interaction score is used to characterize the at least one target object.

36. The method of claim 1, wherein the eliminating (E) comprises:
i) clustering the plurality of test objects, thereby assigning each test object in the plurality of test objects to a respective cluster in a plurality of clusters, and
ii) eliminating a subset of test objects from the plurality of test objects based at least in part on a redundancy of test objects in individual clusters in the plurality of clusters.

37. The method of any one of claim 31, 32, or 36 wherein clustering the plurality of test objects is performed using a density-based spatial clustering algorithm, a divisive clustering algorithm, an agglomerative clustering algorithm, a k-means clustering algorithm, a supervised clustering algorithm, or ensembles thereof.

38. The method of claim 1, wherein the eliminating (E) comprises:
ranking the plurality of test objects based on the instance of the plurality of predictive results, and
removing from the plurality of test objects those test objects in the plurality of test objects that fail to have a corresponding predictive result that satisfies a threshold cutoff.

39. The method of claim 38, wherein the threshold cutoff is a top threshold percentage.

40. The method of claim 39, wherein the top threshold percentage is the top 90 percent, the top 80 percent, the top 75 percent, the top 60 percent, or the top 50 percent of the plurality of predictive results.

41. The method of any one of claims 1-40, wherein each instance of the eliminating (E) eliminates between one tenth and nine tenths of the test objects in the plurality of test objects.

42. The method of any one of claims 1-40, wherein each instance of the eliminating (E) eliminates between one quarter and three quarters of the test objects in the plurality of test objects.

43. The method of any one of claims 1-42, wherein the at least one target object is a single target object and the applying, for each respective test object in a subset of test objects from the plurality of test objects, to the respective test object and target object to obtain a corresponding target result B) comprises:
i) obtaining spatial coordinates for the target object;
ii) modeling the respective test object with the target object in each pose of a plurality of different poses, thereby creating a plurality of voxel maps, wherein each respective voxel map in the plurality of voxel maps comprises the test object in a respective pose in the plurality of different poses;
iii) unfolding each voxel map in the plurality of voxel maps into a corresponding vector, thereby creating a plurality of vectors, wherein each vector in the plurality of vectors is the same size;
iv) inputting each respective vector in the plurality of vectors to the target model, wherein the target model includes (a) an input layer for sequentially receiving the plurality of vectors, (b) a plurality of convolutional layers, and (c) a scorer, wherein
the plurality of convolutional layers includes an initial convolutional layer and a final convolutional layer,
each layer in the plurality of convolutional layers is associated with a different set of weights,
responsive to input of a respective vector in the plurality of vectors, the input layer feeds a first plurality of values into the initial convolutional layer as a first function of values in the respective vector,
each respective convolutional layer, other than the final convolutional layer, feeds intermediate values, as a respective second function of (a) the different set of weights associated with the respective convolutional layer and (b) input values received by the respective convolutional layer, into another convolutional layer in the plurality of convolutional layers, and
the final convolutional layer feeds final values, as a third function of (a) the different set of weights associated with the final convolutional layer and (b) input values received by the final convolutional layer, into the scorer;
v) obtaining a corresponding plurality of scores from the scorer, wherein each score in the corresponding plurality of scores corresponds to the input of a vector in the plurality of vectors into the input layer; and
vi) using the plurality of scores to compute the corresponding target result.

44. The method of claim 43, wherein the scorer comprises a plurality of fully-connected layers and an evaluation layer, and wherein a fully-connected layer in the plurality of fully-connected layers feeds into the evaluation layer.

45. The method of claim 43, wherein the scorer comprises a decision tree, a multiple additive regression tree, a clustering algorithm, principal component analysis, a nearest neighbor analysis, a linear discriminant analysis, a quadratic discriminant analysis, a support vector machine, an evolutionary method, a projection pursuit, and ensembles thereof.

46. The method of claim 43, wherein each vector in the plurality of vectors is a one-dimensional vector.

47. The method of claim 43, wherein the plurality of different poses comprises 2 or more poses, 10 or more poses, 100 or more poses, or 1000 or more poses.

48. The method of claim 43, wherein the plurality of different poses is obtained using a docking scoring function in one of markup chain Monte Carlo sampling, simulated annealing, Lamarckian Genetic Algorithms, or genetic algorithms.

49. The method of claim 43, wherein the plurality of different poses is obtained by incremental search using a greedy algorithm.

50. The method of claim 43, wherein the using the plurality of scores to compute the corresponding target result comprises taking a measure of central tendency of the plurality of scores.

51. The method of claim 43, wherein the using the plurality of scores to compute the corresponding target result comprises using the plurality of scores to characterize the respective test object comprises taking a weighted average of the plurality of scores.

52. The method of claim 43, wherein a respective convolutional layer in the plurality of convolutional layers has a plurality of filters and wherein each filter in the plurality of filters convolves a cubic input space of N³ with stride Y, wherein N is an integer of two or greater and Y is a positive integer.

53. The method of claim 52, wherein the different set of weights associated with the respective convolutional layer are associated with respective filters in the plurality of filters.

54. The method of claim 43, wherein the scorer comprises a plurality of fully-connected layers and a logistic regression cost layer and wherein a fully-connected layer in the plurality of fully-connected layers feeds into the logistic regression cost layer.

55. A computer system for reducing a number of test objects in a plurality of test objects in a test object dataset, the computer system comprising:
one or more processors;
a memory; and
one or more programs, wherein the one or more programs are stored in the memory and are configured to be executed by the one or more processors, the one or more programs including instructions for:
A) obtaining, in electronic format, the test object dataset;
B) applying a target model, for each respective test object in a subset of test objects from the plurality of test objects, to the respective test object and at least one target object to obtain a corresponding target result, thereby obtaining a corresponding subset of target results;
C) training a predictive model in an initial trained state using at least i) the subset of test objects as independent variables and ii) the corresponding subset of target results as dependent variables, thereby updating the predictive model to an updated trained state;
D) applying the predictive model in an updated trained state to the plurality of test objects thereby obtaining an instance of a plurality of predictive results;
E) eliminating a portion of the test objects from the plurality of test objects based at least in part on the instance of the plurality of predictive results; and
F) determining whether one or more predefined reduction criteria are satisfied, wherein, when the one or more predefined reduction criteria are not satisfied, the method further comprises:
(i) applying the target model, for each respective test object in an additional subset of test objects from the plurality of test objects, to the respective test object and at least one target object to obtain a corresponding target result, thereby obtaining an additional subset of target results, wherein the additional subset of test objects is selected at least in part on the instance of the plurality of predictive results;
(ii) updating the subset of test objects by incorporating the additional subset of test objects into the subset of test objects;
(iii) updating the subset of target results by incorporating the additional subset of target results into the subset of target results;
(iv) modifying, after the updating (ii) and the updating (iii), the predictive model by applying the predictive model to at least 1) the subset of test objects as a plurality of independent variables of the predictive model and 2) the corresponding subset of target results as a corresponding plurality of dependent variables of the predictive model, thereby providing the predictive model in an updated trained state; and
(v) repeating the applying (D), eliminating (E), and determining (F), wherein the plurality of test objects, before application of an instance of the eliminating E), comprises at least 100 million test objects.

56. A non-transitory computer readable storage medium and one or more computer programs embedded therein, the one or more computer programs comprising instructions which, when executed by a computer system, cause the computer system to perform a method for reducing a number of test objects in a plurality of test objects in a test object dataset comprising:
A) obtaining, in electronic format, the test object dataset;
B) applying a target model, for each respective test object in a subset of test objects from the plurality of test objects, to the respective test object and at least one target object to obtain a corresponding target result, thereby obtaining a corresponding subset of target results;
C) training a predictive model in an initial trained state using at least i) the subset of test objects as independent variables and ii) the corresponding subset of target results as dependent variables, thereby updating the predictive model to an updated trained state;
D) applying the predictive model in an updated trained state to the plurality of test objects thereby obtaining an instance of a plurality of predictive results;
E) eliminating a portion of the test objects from the plurality of test objects based at least in part on the instance of the plurality of predictive results; and
F) determining whether one or more predefined reduction criteria are satisfied, wherein, when the one or more predefined reduction criteria are not satisfied, the method further comprises:
(i) applying the target model, for each respective test object in an additional subset of test objects from the plurality of test objects, to the respective test object and at least one target object to obtain a corresponding target result, thereby obtaining an additional subset of target results, wherein the additional subset of test objects is selected at least in part on the instance of the plurality of predictive results;
(ii) updating the subset of test objects by incorporating the additional subset of test objects into the subset of test objects;
(iii) updating the subset of target results by incorporating the additional subset of target results into the subset of target results;
(iv) modifying, after the updating (ii) and the updating (iii), the predictive model by applying the predictive model to at least 1) the subset of test objects as a plurality of independent variables of the predictive model and 2) the corresponding subset of target results as a corresponding plurality of dependent variables of the predictive model, thereby providing the predictive model in an updated trained state; and
(v) repeating the applying (D), eliminating (E), and determining (F), wherein the plurality of test objects, before application of an instance of the eliminating E), comprises at least 100 million test objects.
